(19) United States
(12) Patent Application Publication
(10) Pub. No.: US 2023/0085440 A1
(43) Pub. Date: Mar. 16, 2023

(54) METHODS OF PREVENTING AND TREATING PAIN AND ASSOCIATED SYMPTOMS

(71) Applicant: Ethismos Research, Inc., Cambridge, MA (US)

(72) Inventors: Anthony Alexander McKinney, Boston, MA (US); Robert H. Dworkin, Pittsford, NY (US)

(73) Assignee: Ethismos Research, Inc., Cambridge, MA (US)

(21) Appl. No.: 17/899,107

(22) Filed: Aug. 30, 2022

Related U.S. Application Data
(60) Provisional application No. 63/239,059, filed on Aug. 31, 2021.

(51) Int. Cl. A61K 31/403; A61P 25/04

(52) U.S. Cl. CPC A61K 31/403; A61P 25/04

(57) ABSTRACT

The disclosure provides a method of prophylaxis or reduction in the acute to subacute pain transition and the acute to chronic pain transition in a patient in need thereof, comprising administering an effective amount of a novel multimodal compound, amitifadine, in free or pharmaceutically acceptable salt form following the initiation of a painful stimulus, for example surgery.

### RELATED APPLICATIONS

This application claims the benefit of priority under 35 U.S.C. § 119(e) to U.S. Provisional Application No. 63/239,059, filed Aug. 31, 2021, which is incorporated herein by reference in its entirety.

### BACKGROUND

The International Association for the Study of Pain (IASP) has defined chronic pain as pain that lasts or recurs for more than three months (Treede RD, et al. A classification of chronic pain for ICD-11. Pain. 2015 June;156(6):1003-1007). Acute pain tends to arise in response to a painful stimulus either planned such as surgery or as a result of an inadvertent event such as a slip or fall that results in a slipped lower vertebral disc leading to acute (pain for up to 4 weeks) or subacute (pain for between 4 and 12 weeks) phases of low back pain. Acute and subacute pain are generally treatable with conventional pain medications and usually resolve once the injury heals or the painful stimulus is removed. Chronic pain, on the other hand, may persist over a long period, and may be quite different from acute pain, both in etiology and in response to treatment with conventional pain medications such as opioids or non-steroidal anti-inflammatory drugs. Chronic pain is frequently accompanied by mood disorders such as depression or anxiety.

Several prevalent chronic pain conditions are the result of a prior state of acute pain that transitioned into a chronic state. One such example of this transition is persistent post-surgical pain or PPSP (Kehlet H, et al. Persistent postsurgical pain: risk factors and prevention. Lancet. 2006 May 13;367(9522):1618-25.). PPSP is typically neuropathic pain (pain caused by a lesion or disease of the somatosensory nervous system), a highly prevalent complication following surgical procedures. Rates vary by surgical type. For example, cesarean section rates are in the 15% range whereas post-thoracotomy rates rise to the 60-70% range (Richebé P, et al. Persistent Postsurgical Pain: Pathophysiology and Preventative Pharmacologic Considerations. Anesthesiology. 2018 September;129(3):590-607.). PPSP rates at 1 year post-breast surgery are 40-50%. Across all surgeries the average rate of PPSP during the 3 to 36 months post-surgery is approximately 40% (Johansen A, et al. Persistent postsurgical pain in a general population: Prevalence and predictors in the Tromso study. Pain. 2012 July;153(7):1390-1396.). According to Kehlet (ibid.) the management and prevention of postsurgical persistent pain remain inadequate. No medication has been approved for reduction or prevention of PPSP. If the annual rate of surgeries in the U.S. is 50 million (NQF-Endorsed Measures for Surgical Procedures, 2015-2017 FINAL REPORT APRIL 20, 2017. National Quality Forum. www.qualityforum.org), PPSP would be expected to affect approximately 20 million Americans each year.

A second example of a pain condition involving this characteristic transition from acute to chronic pain or subacute to chronic is low back pain. In a typical presentation, a patient experiencing subacute low back pain for 4-12 weeks then goes on to develop sustained or chronic low back pain (CLBP). CLBP is the most frequent type of chronic pain in the US, affecting over 13% of U.S. adults (32 million) (Shmagel A, et al. Epidemiology of Chronic Low Back Pain in US Adults: Data From the 2009-2010 National Health and Nutrition Examination Survey. Arthritis Care Res (Hoboken). 2016 November; 68(11):1688-1694.).

In addition to the neuropathic form of chronic pain and chronic lower back pain mentioned above, other forms of chronic pain include chronic musculoskeletal pain, fibromyalgia, and related central sensitization conditions, as well as visceral pain.

A need remains for additional therapies that inhibit or even prevent transition from acute pain to subacute pain or even further from acute or subacute to chronic pain, as well as for therapies for treating chronic pain.

### BRIEF SUMMARY

Some aspects of the present disclosure are directed to a method of treating a patient experiencing pain or expecting to experience pain, comprising administering to the patient, on a substantially daily basis, a therapeutically effective amount of amitifadine, (+)-1-(3,4-dichlorophenyl)-3-azabicyclo[3.1.0]hexane (IUPAC: (1R,5S)-(+)-1-(3,4-dichlorophenyl)-3-azabicyclo[3.1.0]hexane), or a pharmaceutically acceptable salt thereof, wherein administration of amitifadine is administered daily for a time period comprising 0-3 weeks prior to onset of the pain and about 4 to about 52 weeks after the onset of the pain.

These methods include inhibiting or preventing a transition from pain that is acute (i.e., clinically meaningful pain that lasts up to 4 weeks) to a pain that becomes chronic (i.e., clinically meaningful pain that lasts 12 weeks or longer). The methods may entail inhibition or preventing a first transition from pain that is acute to pain that is subacute pain (i.e., clinically meaningful pain that lasts after 4 weeks and up to 12 weeks). If the first transition does occur, the methods may be effective in inhibiting or preventing a second transition from subacute pain to chronic pain.

In some embodiments, amitifadine is administered substantially daily beginning about 1-3 weeks prior to the onset of pain or a stimulus that causes pain and continued for a time period comprising about 4 weeks after the onset of pain.

In some embodiments, amitifadine is administered substantially daily beginning about 1-3 weeks prior to the onset of pain or a stimulus that causes pain and continued for a time period comprising about 12 weeks after the onset of pain.

In some embodiments, amitifadine is administered substantially daily beginning about 2 weeks prior to the onset of pain or a stimulus that causes pain. In other embodiments, amitifadine is initiated substantially concomitantly with the onset of the pain or a stimulus that causes the pain.

One such method is directed to inhibiting or preventing the first transition, namely the acute to subacute pain transition, which comprises administering to a patient in need thereof, following the onset of acute pain or initiation of a stimulus that causes acute pain such as surgery or trauma, an effective amount of amitifadine, (+)-1-(3,4-dichlorophenyl)-3-azabicyclo[3.1.0]hexane, or salt thereof, wherein inhibition or prevention of the transition from acute pain to subacute pain is measured by time to reduction in or loss of clinically meaningful (also referred to herein as clinically relevant) acute/subacute pain in the patient during the first 3 months following the onset of acute pain or the stimulus that causes acute pain, e.g., surgery, defined as the time needed during the 3 months following acute pain onset for the pain to decrease to <3 on a 0-10 point numerical rating scale (NRS) for the weekly mean of average daily pain intensity, as compared to a placebo (i.e., compared to a patient not receiving amitifadine in a comparable presentation). In some embodiments, amitifadine is administered 2 times daily, such as orally, for substantially the entirety of 3 month period. In some embodiments, administration of amitifadine is ceased at that point.

In this method, the inhibition or prevention of the transition to subacute pain is measured as a function of time, i.e., shortening of the time necessary to achieve a certain pain threshold. For example, a patient who was not treated with amitifadine (placebo) during the first 3 months following the onset of acute pain or the stimulus that causes acute pain might achieve a pain level of <3 by 112 days. In contrast, a patient treated with amitifadine during the first 3 months following the onset of acute pain or the stimulus that causes acute pain might achieve a pain level of <3. Amitifadine treatment may be considered successful if the treated patient achieves the low pain threshold (signifying no transition to subacute pain) by 21 days, or sooner than a patient who does not receive the treatment with amitifadine. As described herein, beneficial effects of the drug may inure to a patient's benefit in that the NRS score may remain below 3 (indicative of an absence of clinically meaningful pain) even after cessation of the treatment as of the end of month 3 (e.g., without taking amitifadine in months 4-6).

Even if a patient misses the first endpoint and the transition from acute pain to subacute pain does occur, e.g., hits a NRS of 4, the multi-modal effects of the drug, including mesolimbic dopamine restoration, might prove to drive down the NRS to below 3 by 6 months following the onset of the pain (also referred to herein as a second endpoint). Therefore, even if the treatment with amitifadine is unsuccessful in preventing transition to subacute pain for any given patient, it might be effective in preventing a transition to chronic pain (as measured by an NRS of less than 3 at the 6 month point).

Accordingly, another such method is directed to inhibiting or preventing the acute to chronic pain transition comprising administering to a patient in need thereof, following the onset of acute pain or initiation of a stimulus that causes acute pain such as surgery or trauma, an effective amount of amitifadine, (+)-1-(3,4-dichlorophenyl)-3-azabicyclo[3.1.0]hexane, or salt thereof, wherein inhibition or prevention of the transition from acute pain to chronic pain is measured by clinically important persistent post-surgical pain at 6-months, defined as average pain intensity during the previous week on a 0-10 point numerical rating scale (NRS) at 6-months, as compared to a placebo (i.e., compared to a patient not receiving amitifadine in a comparable presentation). This method is based on a negative value in that it determines if the patient has arrived at the destination of their “journey” completed at 6 months, namely whether their pain intensity is greater than or equal to 3 on an NRS. If a treated patient's NRS score is greater than or equal to 3, the transition to chronic pain has occurred. If on the other hand, the treated patient's NRS score is below 3, the treatment is considered to have been effective in inhibiting or preventing the transition from acute pain transition to chronic pain.

In some embodiments, the patient is experiencing or expecting to experience pain as a result of surgery, and the methods may be effective in preventing a transition from acute pain to chronic pain, which in this specific context is referred to as persistent post-surgical pain (PPSP). In some embodiments, the surgery is soft tissue surgery such as breast surgery or cardiovascular surgery. In some embodiments, the surgery is orthopedic surgery, e.g., joint replacement surgery such as hip, knee or shoulder replacement surgery.

In some embodiments, amitifadine is administered substantially daily beginning about 2 to 3 weeks prior to surgery up to about a day prior to surgery and continued beginning about a day after surgery. Due to its unique, multi-modal actions, administration of amitifadine prior to surgery may ameliorate different constellations of symptoms during the extended time prior to surgery. As a monoamine reuptake inhibitor with a brain half-life approximately 3x longer than its plasma half-life, amitifadine does not operate according to plasma drug levels. For example, amitifadine is still active in the brain up to two days after the last oral dose. Thus, even though a surgical patient is Non Per Os (NPO) or nothing by mouth the morning of surgery, amitifadine is still pharmacologically active from the dose the previous evening. As used herein, the term “substantially daily” and “substantially on a daily basis” refer to a brief cessation of treatment, e.g., a one- or two-day interruption in amitifadine treatment on a prescribed basis and/or patient omission.

By way of example, not every single woman experiencing breast surgery is predicted to still have chronic pain 3 to 6 months following surgery, just approximately half. Given the probabilities, if amitifadine reduces the rate vs. placebo, the disclosed methods may be considered successful.

In some embodiments, at least one effective dose of amitifadine is administered daily over the course of at least 3 weeks during this time period. In embodiments wherein the stimulus or onset of acute pain was planned, administration of amitifadine may begin suitably prior to the initiation of the stimulus, e.g., surgery. In some embodiments, administration of the amitifadine may commence about 1 to 3 weeks prior to the initiation of the stimulus. In some embodiments, the administration of amitifadine may continue for another 2 weeks, another 4 weeks, another 12 weeks, another 24 weeks, another 36 weeks, another 48 weeks, another 52 weeks, or even longer. In embodiments wherein the acute pain stimulus is unplanned, administration of amitifadine may commence immediately thereafter, such as on day zero, e.g., the day of injury.

In some embodiments, the person is anticipating or experiencing surgery and has a history of, or has symptoms of pain catastrophizing, i.e., a maladaptive response to pain characterized by an experience of heightened pain intensity, increased disability, and difficulty disengaging from pain (Seminowicz D, et al. Cortical responses to pain in healthy individuals depends on pain catastrophizing. Pain. 2006 February;120(3):297-306.). Pain catastrophizing affects brain areas distinct from other affective disorders like depression and anxiety. In other words, pain does not automatically infer a co-diagnosis of depression or anxiety or pain catastrophization or both or all three or all four. Pain catastrophizing is a major factor contributing to the patient's pre-operative emotional functional state and whether the patient will develop persistent post-surgical pain (Khan R, et al. Catastrophizing: a predictive factor for post-operative pain. Am J Surg. 2011 January;201(1):122-31.). Thus, inhibition or even prevention of persistent post-surgical pain (PPSP) may be achieved by pre-operative inhibition of pain catastrophizing by pre-operative administration of amitifadine in an effective amount (compared to patients not receiving amitifadine for a comparable period).

In some embodiments, a patient may be anticipating or has experienced surgery and has a history of, or is experiencing depression or anxiety, or both depression and anxiety that contribute to patient's pre-operative emotional functional state. Patients with impaired emotional function, i.e., depression, anxiety, or both, pre-operatively, have worsened chronic pain outcomes (Katz J, et al. Risk factors for acute pain and its persistence following breast cancer surgery. Pain. 2005 Dec 15;119(1-3):16-25.). Thus, inhibition or even prevention of persistent post-surgical pain (PPSP) may be achieved by pre-operative inhibition of depression or anxiety or both by pre-operative administration of amitifadine in an effective amount (compared to patients not receiving amitifadine for a comparable period).

In some embodiments, a patient may be anticipating surgery and is experiencing preoperative pain that contributes to the patient's pre-operative emotional functional state. Patients with pain and impaired emotional function prior to surgery have worsened chronic pain outcomes (Andersen KG, et al. Predictive factors for the development of persistent pain after breast cancer surgery. Pain. 2015 Dec;156(12):2413-2422.). Thus, inhibition or even prevention of persistent post-surgical pain (PPSP) may be achieved by pre-operative reduction of pain by pre-operative administration of amitifadine in an effective amount (compared to patients not receiving amitifadine for a comparable period).

In some embodiments, the patient experiencing severe acute pain (NRS >4/10) is a post-surgical patient. Amitifadine has multi-modal analgesic properties from effects on four descending pathways, thus a post-surgical patient in severe acute pain (NRS >4/10) may derive particular benefit receiving amtifadine in a therapeutically effective amount. The inhibition or prevention of the transition from acute to chronic pain may be achieved by reducing post-surgical pain (PSP) (compared to patients not receiving amitifadine for a comparable period). The method results in reduction of PPSP defined as pain intensity on a 0-10 point numerical rating scale (NRS) at 6-months post-surgery compared to placebo.

In some embodiments, the patient experiencing severe acute pain (NRS >4/10) is a post-surgical patient and the inhibition or prevention of the onset of the acute to subacute transition may be achieved by reducing post-surgical pain (PSP) (compared to patients not receiving amitifadine for a comparable period). The subacute period is the intermediate time, 4-weeks to 12-weeks post-injury. The method results in a reduction in time to recovery defined as post-surgical pain <3 on a 0-10 NRS, among patients treated with amitifadine, compared to patients not receiving amitifadine.

In some embodiments, patient is experiencing pain as a result of non-surgical trauma such as a vehicular accident, a sports injury, a slip or fall, or a military injury.

Concomitant with the inhibition or prevention of the transition from acute to chronic pain (including the first and second transitions), the present methods may ameliorate one or more different types of associated symptoms, including allodynia, hyperalgesia, and pain-associated affective (emotional) symptoms such as aversion, anhedonia, anxiety, dysphoria, and/or depression as well as pain-associated executive dysfunction symptoms such as (pain) catastrophizing, cognitive impairment, inattention, increase in impulsivity, and/or loss of motivation.

In some embodiments the patient may present symptoms of cognitive impairment due to pain. In these embodiments, the present methods may also mitigate or reduce cortical gray matter loss as a result of the acute to chronic pain transition or the acute to subacute transition.

Without intending to be bound by any particular theory of operation, amitifadine is believed to exert its therapeutic effects in terms of inhibiting or preventing the acute to chronic pain transition via 4 distinct modes of action that operate during any one or more of three distinct milieus of time. This transition, also referred to herein as pain chronification, results in a state of chronic pain, namely pain that persists 3-6 months post-surgery. The modes of action are explained in the specific context of embodiments of the disclosure, namely treatment of patients having surgery (e.g., breast surgery such as a lumpectomy/mastectomy), a prototypic cause of acute pain, and the inhibition or prevention of a transition from acute pain to chronic pain, which in the specific context of surgery, is referred to herein as persistent post-surgical pain (PPSP).

The first time period occurs prior to surgery. Amitifadine administration may, in some embodiments, be initiated 1 or 2 weeks prior to surgery. Patients who are susceptible to pain chronification may manifest a constellation of pre-operative psychosocial symptoms precipitated by the impending surgery: anxiety, depression, pain, and pain catastrophizing (rumination on pain; magnification on symptoms of pain; having a feeling of helplessness on the pain). In many patients chronic pain persists for life and is very difficult to treat. No drugs are approved to prevent or treat PPSP.

Due to its rapid onset of action, amitifadine works as an analgesic, anti-depressant, anti-anxiety agent, and anti-pain catastrophization agent during the pre-op period when greater psychosocial distress places the vulnerable patient at risk for PPSP. Four modes of action are in play during this stage, namely inhibition of serotonin (5-HT) reuptake, inhibition of norepinephrine (NE) reuptake, inhibition of dopamine (DA) reuptake, and serotonin 2c (5-HT2c) receptor partial agonism. Reuptake inhibition at the 5-HT, NE, and DA transporters, SERT, NET, and DAT, respectively, causes modulation of these three descending pathways resulting in analgesia, and reductions in anxiety, and depression symptoms. Serotonin 2c partial agonism at specific forebrain circuits also causes analgesia, affective (emotional) symptom relief, as well as reductions in pain catastrophization symptoms.

The second time period occurs during the initial 4 weeks after surgery, which is when the patient is experiencing maximum acute pain. Younger patients, patients having more invasive surgery (e.g., lumpectomy plus axillary exploration), patients receiving adjunctive chemotherapy or radiation, and those with severe post-operative pain are more likely to undergo a transition from acute to chronic pain. Amitifadine therapy during this time period may attenuate the pain by minimizing pain amplification caused by accompanying anxiety, depression, and pain catastrophization. This in turn may ameliorate the pain and emotional/cognitive symptoms linked to pain that trigger the long-term transition to chronic pain and therefore, inhibit or prevent onset of pain chronification.

The same modes of action mentioned above, 5-HT, NE, and DA reuptake inhibition, are in play in this time period as well. They each continue to modulate the three descending pathways to reduce pain and affective symptoms. Similarly, 5-HT2c partial agonism continues to modulate 5-HT2cR-specific forebrain circuits, resulting in analgesia, reduction of pain catastrophization, and modulation of affective symptoms.

To the extent the patient is receiving concomitant opioid therapy, since amitifadine is a non-opioid analgesic, it may also be effective to reduce opioid dose (opioid-sparing). By using multi-modal activity on severe pain, anxiety, depression, hyperalgesia, and pain catastrophization, amitifadine may further reduce post-operative pain and reduce acute pain medication (e.g., opioid) requirements thereby reducing potential side effects and hastening recovery while reducing the potential for pain chronification.

A third time period occurs 4-12 weeks after surgery. Lingering pain is intrusive and may cause cognitive impairment (e.g., inattention, impulsivity) and loss of motivation. Constant pain is also aversive (unpleasant) leading to anxiety and depression (emotional impairment). The emotional/cognitive center in the forebrain, the prefrontal cortex (PFC), signals this distress to the reward center (nucleus accumbens or NAc) in the midbrain. Pain causes a reduction in dopamine (DA) levels in the mid-brain, leading to allodynia (pain from stimuli that normally does not cause pain), aversion, anhedonia (inability to perceive pleasure), depression, and impaired motivation. Allodynia and these emotional/cognitive symptoms may signal that the acute to chronic pain transition has begun. Accordingly, a related aspect of the disclosure is directed to a method for activating pyramidal neurons of the prefrontal cortex (PFC) in a patient experiencing pain or expecting to experience pain, comprising administering to the patient, on a substantially daily basis, a therapeutically effective amount of amitifadine, (+)-1-(3,4-dichlorophenyl)-3-azabicyclo[3.1.0]hexane (IUPAC: (1R,5S)-(+)-1-(3,4-dichlorophenyl)-3-azabicyclo[3.1.0]hexane), or a pharmaceutically acceptable salt thereof, wherein administration of amitifadine is administered daily for a time period comprising 0-3 weeks prior to onset of the pain and about 4 to about 52 weeks after the onset of the pain.

Continuing amitifadine therapy throughout this time period (e.g., continued through 12 weeks post-surgery), has several beneficial effects. First, it may result in 5-HT and NE reuptake inhibition each continuing to modulate the serotonin and norepinephrine descending pathways to reduce pain and affective (emotional) symptoms. Similarly, DA reuptake inhibition, which causes tonic (constant) DA increase, and therefore ameliorate allodynia, aversion, anhedonia, depression, hyperalgesia, and loss of motivation. Amitifadine may also increase serotonin 2c partial agonism (stimulation of the 5-HT2c receptor). As mentioned previously, this activity has prefrontal analgesic effects and may reduce cognitive impairment (decrease impulsivity and inattention; improve motivation) and provide further improvements in allodynia, aversion, anhedonia, anxiety, depression, and hyperalgesia while treating pain catastrophization. Importantly, 5-HT2c receptor partial agonism has a separate unique function, namely inhibition of phasic (or rapid/burst) dopamine release in the midbrain (e.g., nucleus accumbens). This activity may serve to reduce the abuse potential of amitifadine.

Amitifadine's multi-modal, non-rewarding pharmacology is uniquely suited to treat these interrelated but different constellations of symptoms. Therefore, it may be singularly effective in inhibiting the transition from acute to chronic pain, especially in vulnerable patients. Patients are benefited by avoiding lifelong misery and potential exposure to alternative and less effective therapies with attendant risky side effects (e.g., opioids).

The beneficial multi-modal effects of amitifadine are summarized in the following table.

| Pharmacology | Amitifadine Activity |
|---|---|
| Serotonin (5-HT) Reuptake Inhibition | Analgesia, Antidepressant, Antianxiety |
| Norepinephrine (NE) Reuptake Inhibition | Analgesia, Antianxiety, Improves Cognition, Treats |
| | Inattention, Improves Motivation |
| Dopamine (DA) Reuptake Inhibition | Analgesia (reduces Allodynia), Reduces Aversion |
| | (unpleasantness of pain), Reduces Anhedonia (inability to |
| | feel pleasure), Improves Cognition, Improves Motivation, |
| | Reduces Hyperalgesia |
| Serotonin 2c (5-HT2c) Partial Agonism | Analgesia (reduces Allodynia), Reduces Aversion, Reduces |
| | Depression, Treats Pain Catastrophization, Reduces |
| | Impulsivity, Reduces Inattention; Prevents Phasic or Burst |
| | (rewarding) DA release |

Another aspect of the present disclosure is directed to a method of treating a patient experiencing chronic pain, comprising administering to the patient, on a daily basis, a therapeutically effective amount of amitifadine, (+)-1-(3,4-dichlorophenyI)-3-azabicyclo[3.1.0]hexane (IUPAC: (1R,5S)-(+)-1-(3,4-dichlorophenyl)-3-azabicyclo[3.1.0]hexane), or a pharmaceutically acceptable salt thereof.

In some embodiments, amitifadine is administered daily for about 1— 52, e.g., about 24 weeks. In some embodiments, the patient is experiencing musculoskeletal pain such as chronic lower back pain. In some embodiments, the patient is experiencing neuropathic pain such as chronic postherpetic neuralgia. In some embodiments, the patient is experiencing centralized pain such as chronic fibromyalgia. In some embodiments, the patient is experiencing visceral pain such as chronic pelvic pain.

In some embodiments, the patient has a history of, or has symptoms of pain catastrophization comprising a maladaptive response to pain characterized by an experience of heightened pain intensity, increased disability, or difficulty disengaging from pain or a belief that pain will intensify and cannot be ameliorated. In some embodiments, the patient has a history of, or is experiencing depression or anxiety. In some embodiments, the patient presents with symptoms of cognitive impairment. In some embodiments, the patient is experiencing anhedonia.

In some embodiments, the patient is an adult female.

In some embodiments of any of the disclosed methods, the patient is an adult female. According to a large federal study women represent the majority (56%) of the 50 million people in the U.S. afflicted with chronic pain, and over 11 million (58%) of the nearly 20 million in the U.S. afflicted with high impact, disabling chronic pain (Dahlhamer J, et al. Prevalence of Chronic Pain and High-Impact Chronic Pain Among Adults - United States, 2016. MMWR Morb Mortal Wkly Rep. 2018 Sep 14;67(36):1001-1006.). Depression is known to be highly co-morbid with chronic pain and women experience depression at a rate twice that of men (Van de Velde S, et al. Gender differences in depression in 23 European countries. Cross-national variation in the gender gap in depression. Soc Sci Med. 2010 Jul;71(2):305-313.). Therefore, the present methods may benefit women from an additional standpoint of ameliorating depression associated with the transition from acute to chronic pain or the transition from acute to subacute pain.

In some embodiments, the patient is a woman of child-bearing age. In these embodiments, the present methods exploit the interactions between ovarian hormones (estradiol, progesterone) and dopamine. Estradiol is involved with mesolimbic dopamine circuitry to amplify motivational processes. Since amitifadine has multiple dopaminergic actions, it is believed that the present methods will achieve synergistic results in cycling women.

Another aspect of the present disclosure is directed to a method for activating neurons of the prefrontal cortex (PFC) in a patient experiencing pain or expecting to experience pain, comprising administering to the patient, on a substantially daily basis, a therapeutically effective amount of amitifadine, (+)-1-(3,4-dichlorophenyl)-3-azabicyclo[3.1.0]hexane (IUPAC: (1R,5S)-(+)-1-(3,4-dichlorophenyl)-3-azabicyclo[3.1.0]hexane), or a pharmaceutically acceptable salt thereof, wherein administration of amitifadine is administered daily for a time period comprising 0-3 weeks prior to onset of the pain and about 4 to about 52 weeks after the onset of the pain.

In some embodiments of any of the disclosed methods, amitifadine may be administered in the form of a pharmaceutically acceptable active salt e.g., hydrochloride salt. The amitifadine and compositions in which the amitifadine may be substantially free of the corresponding (−) enantiomer, (−)-1-(3,4-dichlorophenyl)-3-azabicyclo[3.1.0]hexane. In addition to being enantiomeric, amitifadine or (+)-1-(3,4-dichlorophenyl)-3-azabicyclo[3.1.0]hexane exists in at least three polymorphic forms, labeled herein polymorphs A, B and C. The polymorphs may be used in pharmaceutical compositions in combination or in forms that are substantially free of one or more of the other polymorphic forms.

In some embodiments of any of the disclosed methods, amitifadine is administered orally. In some embodiments, amitifadine is administered once or twice on a substantially daily basis. In some embodiments, amitifadine is administered in a daily dosage of about 10 to about 300 mg and in some embodiments from about 10 mg to about 200 mg. In some embodiments of any of the disclosed methods, the patient is receiving concomitant opioid therapy.

The disclosed methods offer several advantages over current therapeutic modalities. For example, opioids are effective for pain but have abuse liability, cause hyperalgesia, and cause both anxiety and depression. Neither anticonvulsants nor antidepressants approved for chronic pain have dopaminergic activity. In contrast, amitifadine has 4 discrete pharmacological effects in one molecule: 5-HT, NE, and DA reuptake inhibition, and 5-HT2c partial agonism. This multi-modal pharmacology produces analgesia, improves affective symptoms associated with pain, and improves executive function and cognition. These properties enable treatment of pre-operative symptoms that increase the risk of chronic pain, as well as the pain-triggered hypodopaminergia-associated affective symptoms that lead to pain chronification post-operatively. Serotonin 2c partial agonism prevents accumbral phasic (burst) dopamine release thereby reducing risk of abuse liability.

### BRIEF DESCRIPTION OF THE DRAWINGS

FIG. 1 is a clinical study timeline.

### DETAILED DESCRIPTION

The following description of preferred embodiments is merely exemplary in nature and is in no way intended to limit this disclosure, its application, or uses.

As used throughout, ranges are used as shorthand for describing each and every value that is within the range. Any value within the range can be selected as the terminus of the range. In the event of a conflict in a definition in the present disclosure and that of a cited reference, the present disclosure controls.

A typical way to classify pain is to consider pain along a continuum of duration. Acute pain, subacute pain, and chronic pain are defined by units of time. Acute pain is pain present for less than 4 weeks. Subacute pain is defined as pain present for greater than 4 weeks but less than 12 weeks. Chronic pain is pain present for 12 weeks or longer (Chou R, et al. Diagnosis and treatment of low back pain: a joint clinical practice guideline from the American College of Physicians and the American Pain Society. Ann Intern Med. 2007 Oct 2;147(7):478-91.).

Another way to classify pain is to use pain severity or intensity as a linear dimension measured on a categorical scale, e.g., “Mild”, “Moderate”, or “Severe”, or a visual analog scale (a continuous point along a 10 centimeter line), or a numerical rating scale (NRS), an 11-point scale where the patient is asked to rate their pain on a scale between 0-10 with 0, “No pain” to 10, “Worst pain imaginable.” (D.C. Turk and R. Melzack (Eds.). Handbook of Pain Assessment. Guilford Press, New York, 1992a. pp. 1-16.). The presence and severity of pain may be combined to consider pain above or below a “clinically meaningful” or moderate pain intensity level (e.g., NRS 3/10) (Jennifer S Gewandter J, et al. Research design considerations for chronic pain prevention clinical trials: IMMPACT recommendations. Pain. 2015 July;156(7):1184-1197.).

The severity of pain and its duration may be combined into a single numeric value, represented by estimating the ‘area under the curve’ (AUC) by plotting the magnitude and duration of pain during treatment and computing the total pain reflected by this plot. Because pain can vary through the day, the patient is asked to rate the pain at the time of responding to a pain questionnaire and also the pain at its worst, least and average during the previous 24 hours. The length of time between two consecutive measurements is multiplied by the average of the ‘worst pain’ AUC (Lydick E, et al. Area under the curve: a metric for patient subjective responses in episodic diseases. Qual Life Res. 1995 February;4(1):41-5.).

(+)−1−(3,4-dichlorophenyl)-3-azabicyclo[3.1.0]hexane, also known as (1R,5S)-1-(3,4-dichlorophenyl)-3-azabicyclo[3.1.0]hexane, also known as amitifadine, is shown as Formula I below.

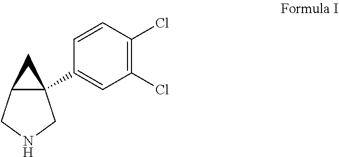 [SMILES: Clc1ccc([C@]23CNC[C@H]2C3)cc1Cl]

“(+)-1-(3,4-dichlorophenyl)-3-azabicyclo[3.1.0]hexane,” “(1R,5S)-1-(3,4-dichlorophenyl)-3-azabicyclo[3.1.0]hexane,” and “amitifadine” are used interchangeably herein. (1R,5S)-1-(3,4-dichlorophenyl)-3-azabicyclo[3.1.0]hexane is an unbalanced triple reuptake inhibitor with the greatest potency towards serotonin (5-HT) reuptake, half as much towards norepinephrine (NE) reuptake, and one eighth towards dopamine (DA) reuptake. (1R,5S)-1-(3,4-dichlorophenyl)-3-azabicyclo[3.1.0]hexane hydrochloride is reported to inhibit the reuptake of [³H] serotonin, [³H]norepinephrine, and [³H]dopamine in human embryonic kidney (HEK) 293 cells expressing the corresponding human recombinant transporters at IC₅₀ values of 12, 23, and 96 nM, respectively. Amitifadine or (+)-1-(3,4-dichlorophenyI)-3-azabicyclo[3.1.0]hexane may be synthesized as described in U.S. Pat. Nos. 6,372,919, 7,098,229, and 9,527,813, each of which is hereby incorporated by reference in their entirety.

Amitifadine, (+)-1-(3,4-dichlorophenyI)-3-azabicyclo[3.1.0]hexane as used herein is substantially free of the corresponding (−) enantiomer, (−)-1-(3,4-dichlorophenyl)-3-azabicyclo[3.1.0]hexane. The term “substantially free of its corresponding (−)-enantiomer” means containing no more than about 5% w/w of the corresponding (−)-enantiomer, preferably no more than about 2% w/w of the corresponding (−)-enantiomer, more preferably no more than about 1% w/w of the corresponding (−)-enantiomer.

In addition to being enantiomeric, multimodal (+)-1-(3,4-dichlorophenyl) azabicyclo[3.1.0]hexane exists in at least three polymorphic forms, known as herein polymorphs A, B and C. The multimodal polymorphs may be used in pharmaceutical compositions in combination or in forms that are substantially free of one or more of the other polymorphic forms.

Polymorphs A, B and C of amitifadine are described in U.S. Pat. Nos. 8,765,801 and 9,770,436. There are key major peaks at given angles in the X-ray powder diffraction (XRPD) patterns for polymorphs A, B and C, which are unique to each given polymorph form. These peaks are present in the XRPD patterns of each of the polymorph forms having a crystal size of about 10 to 40 microns. Any of these major peaks, either alone or in any distinguishing combination, are sufficient to distinguish one of the polymorph forms from the other two polymorph forms.

For polymorph form A, the “° 2θ” angles of these major peaks which characterize polymorph form A are as follows: 17.14; 19.62; 21.96; 24.52; and 26.74. Any of these major peaks, either alone or in any distinguishing combination, are sufficient to distinguish polymorph form A from the other two polymorph forms. For polymorph form B, the “° 2θ” angles of these major peaks which characterize polymorph form B are as follows: 15.58; 17.52; 21.35; 23.04; 25.43; and 30.72. For polymorph form C, the “° 2θ” angles of these major peaks which characterize polymorph form C are as follows: 13.34; 17.64; 20.07; 21.32; 22.97; 24.86; 26.32; and 27.90.

Amitifadine may be in the form of a pharmaceutically acceptable salt. Pharmaceutically acceptable forms may include inorganic and organic acid addition salts such as hydrochloride salts.

Amitifadine reuptake inhibition of 5-HT (12 nM), NE (23 nM), and DA (96 nM) may also be expressed as a reuptake ratio of 1 to 2 to 8. Amitifadine may be referred to as an “unbalanced” triple reuptake inhibitor in reference to the ratio of reuptake inhibition of 5-HT to NE to DA. As described below, this does not relate to 5-HT2c partial agonism.

In addition, amitifadine, is uniquely a partial agonist at the 5-HT2c receptor with a K, =47 nM against [³H]mesulergine, an EC₅₀ value of [³⁵S]guanosine 5-O-(3-thio)triphosphate binding=190 nM, and Eₘₐₓ=52%]. Serotonin 2c receptors (5-HT2cRs) are found throughout the brain and exert inhibitory control of dopamine release. In the mesolimbic area, this function is especially important because Navailles et al., (Navailles S, et al. Region-dependent regulation of mesoaccumbens dopamine neurons in vivo by the constitutive activity of central serotonin2C receptors. J Neurochem. 2006 November; 99(4):1311-9.) demonstrated that ventral tegmental area (VTA) 5-HT2cRs control phasic (burst) release of dopamine at the NAc but not tonic (constant or basal) dopamine release. Thus, control of spike or burst phasic dopamine is maintained under control by 5-H2c agonist activity but tonic levels are undiminished allowing amitifadine DA reuptake to restore mid-brain hypodopaminergic levels without impediment.

In each of the acute to chronic/acute to subacute transitional pain states described herein, changes are observed in patient's emotional and cognitive state that contribute to the high rates of affective (emotional) and cognitive co-morbidities in patients transitioning from acute to chronic pain. Many patients with chronic pain have co-morbid depression. Patients with chronic pain and depression are more likely to have a poor prognosis compared to those with chronic pain alone. For example, Olfson et al. (Olfson M, et al. Service Use Preceding Opioid-Related Fatality. Am J Psychiatry. 2018 Jun 1;175(6):538-544.) found in the last 30 days of life, patients with chronic non-cancer pain but without opioid use disorder who died of opioid overdose were compared to those opioid-related deaths without a chronic pain diagnosis. In the last 30 days of life prior to death due to an opioid overdose, those with a chronic pain diagnosis were significantly more likely to have filled a prescription for opioids (49% vs. 17%, p<0.0001), benzodiazepines (52% vs. 27%, p<0.0001), and antidepressants (39% vs. 19%, p<0.0001) yet diagnoses for opioid use disorder among the same groups during the period were nearly identical and low (4.2% vs. 4.3%, p=0.87) supporting the concept that affective symptoms contribute to poor prognoses in chronic pain.

Chronic pain and depression display similar changes in brain structure and involve overlapping brain systems. As pain develops into a chronic condition, negative emotional states may be accompanied by other emotional (affective) disorders such as anhedonia (inability to perceive pleasure), pain catastrophizing, cognitive deficits, depression, dysphoria (a state of unease or generalized dissatisfaction with life), increase in impulsivity (loss of impulse control), sleep disturbances, and suicide.

Sustained pain triggers the emotional learning and cognitive impairments that underlie the transition to chronic pain. As pain develops into a constant burden, patients in the acute to sub/chronic pain transition develop affective (emotional) and cognitive disturbances. Consistent with these psychological signs of chronic pain, neuroimaging studies have demonstrated changes in brain anatomy and function consistent with injury to brain structures indicative of chronic emotional and cognitive dysfunction associated with the acute to sub/chronic pain transition.

A cardinal sign of the development of the affective symptoms associated with chronic pain is mesolimbic hypodopaminergia or low mid-brain levels of dopamine. Hypodopaminergia is associated with allodynia (pain due to a stimulus that does not normally provoke pain), increase in pain aversion (the unpleasantness of pain), and hyperalgesia (increased pain from a stimulus that normally provokes pain), anhedonia (inability to perceive pleasure), as well as the affective symptoms of pain including anxiety, depression, and dysphoria, and an increase in executive dysfunction including pain catastrophizing, cognitive deficits, increase in impulsivity, and loss of motivation. Mesolimbic hypodopaminergia, affective symptoms, executive dysfunctions, and pain symptoms are characteristic of the transition of acute to chronic pain.

Pain causes a reduction in mesolimbic extracellular dopamine leading to a drop in excitatory activity on indirect Spiny Projection Neurons (iSPNs) in the nucleus accumbens in the mesolimbic region (the “reward” center) that normally are suppressed by dopamine. This leads to increases in allodynia and aversion.

In the mesocortical region, the prefrontal cortex (PFC) provides top-down control of cognitive and emotional processes including executive control, task switching, and response inhibition. In transition of acute to chronic pain, the PFC interacts with the NAc. Over the 1 to 12 months following acute injury, the functional connectivity between these two brain regions becomes fixed. Once the connection becomes fixed, allodynia (pain), anxiety, aversion, anhedonia, behavioral despair/depression, lack of motivation, increases in impulsivity, are recognized as pain/allodynia, aversion, and affective symptoms of chronic pain.

Serotonin 2c receptors are located in high concentrations in long-distance afferent PFC pyramidal glutamatergic neurons projecting to the NAc. These 5-HT2c receptors are located in several PFC subregions including the prelimbic and infralimbic cortices where it is believed that amitifadine 5-HT2c partial agonism facilitated by serotonin neurotransmission may restore iSPN excitability. If confirmed, this 5-HT2c partial agonist-mediated excitatory glutamatergic activity originating in long-distance PFC afferents may ultimately enable treatment of pain/allodynia, aversion, and affective symptoms of chronic pain and reverse chronic neuroplastic changes in the corticolimbic system, and thereby facilitate prevention and treatment of pain chronification.

Thus, in the mesocorticolimbic area, amitifadine has multiple unanticipated and layered antiallodynic effects: (1) Direct 5-HT reuptake inhibition engages serotonin descending pain pathways and NE reuptake inhibition engages norepinephrine descending pain pathways, both types originating in the brainstem. Direct dopamine reuptake inhibition engages dopaminergic descending pain pathways, for example, the spinal trigeminal circuit. Direct action with 5-HT2c partial agonism engages 5-HT2c-specific descending pain pathways (e.g., the ventrolateral orbital cortex or VLO). Activity at each of these four descending pain pathways causes a reduction in pain transmission from the periphery to the brain; (2) Direct DA reuptake inhibition causes tonic (constant) increases in synaptic DA levels to correct pain-induced mesolimbic hypodopaminergia to block pain aversion/allodynia/anhedonia; (3) Mesocortical 5-HT2c partial agonism at 5-HT2cR in deep layer (layer V) long-distance pyramidal neurons in the prefrontal cortex is predicted to excite pyramidal glutamatergic neurons to restore iSPN excitability in the nucleus accumbens and thereby reduce allodynia, aversion, anhedonia, anxiety, and depression; (4) Mesocortical 5-HT2c partial agonism at 5-HT2cR in the PFC reduces impulsivity and is predicted to reduce pain catastrophizing; (5) Mesolimbic 5-HT2c partial agonist activity inhibits phasic (burst) DA activity at VTA and inhibits burst NAc release. Reduction of burst (phasic) NAc dopamine release in the context of complex interactive and unpredictable neurotransmission processes involving dopamine, norepinephrine, and serotonin, globally engenders less risk of euphorigenic potential and less risk of abuse.

A characteristic marker of phasic DA release is locomotor activation and stereotypies, dopamine-driven behaviors manifesting as inability to remain at rest, and focused behavioral stereotypies. In animals these stereotypies manifest as continuous oral behaviors (licking, gnawing, and chewing), focused sniffing, and characteristic repetitive head movements. Behaviors of patients that typify stereotypy in stimulant abusers range from repetitions of single or multiple movements (motor stereotypies) to repetitive, inflexible patterns of attention, emotion, planning, and cognitive loss. Drugs that increase phasic DA release at NAc cause locomotor activation and stereotypies, e.g., amphetamines or cocaine are also rewarding and are often abused (Kuczenski R, et al. Amphetamine, cocaine, and fencamfamine: relationship between locomotor and stereotypy response profiles and caudate and accumbens dopamine dynamics. J Neurosci. 1991 September;11(9):2703-12.).

By simultaneously reducing allodynia/aversion while simultaneously directly increasing mesolimbic synaptic DA levels via reuptake inhibition, the combination of dual DA reuptake inhibition and 5-HT2c partial agonism while applying 4-way descending pathway pain modulation enables therapeutic treatment of allodynia, aversion, hypodopaminergia, and affective symptoms of pain such as anhedonia, anxiety, catastrophization, depression, increase in impulsivity, and loss of motivation thus inhibiting or preventing the acute to chronic pain/acute to subacute pain transition with less risk of addiction or abuse.

Additionally, provided herein are combinatorial multimodal compositions and coordinate treatment means using additional or secondary analgesic or psychotherapeutic agents in combination with amitifadine during the perioperative setting. Representative examples of suitable secondary analgesic or psychotherapeutic drugs for use in the inhibition of acute to subacute pain transition or acute to chronic pain transition in the compositions and methods herein include drugs from the general classes of anesthetics (examples: inhalational, opioid-type, dissociative, benzodiazepine-type, propofol-type), muscle relaxants, drugs from the non-steroidal anti-inflammatory class, acetaminophen, anti-convulsants, anxiolytics, opioid receptor agonists, calcium/sodium channel blockers, a2 receptor agonists, and antidepressants. (See, e.g., R J. Baldessarini in Goodman & Gilman's The Pharmacological Basis of Therapeutics, 11th Edition, Chapters 17 and 18, McGraw-Hill, 2005 for a review).

Various embodiments of the present disclosure are described below in terms of enumerated methods and more specific embodiments thereof.

Provided is a method (Method 1) of inhibiting or preventing the acute to chronic pain transition defined as a reduction or prevention of persistent pain (e.g., post-surgical pain (PPSP) or chronic pain intensity (Clinically Important PPSP) on a 0-10 point numerical rating scale (NRS) at 12- or 24-weeks following the onset of acute pain or initiation of a stimulus that causes acute pain such as surgery or trauma. The method comprises administering to a patient, following the onset of acute pain or initiation of a stimulus that causes acute pain such as surgery or trauma, amitifadine in a dose sufficient to inhibit or even prevent the acute to chronic transition in the patient (e.g., a post-surgical patient) compared to a patient not receiving amitifadine in a comparable presentation.

For purposes of the disclosed methods, causes of acute pain may include surgery, representative examples of which are soft tissue surgery such as shoulder, breast, cardiac and ovarian surgery, and orthopedic surgery such as joint replacement surgery (e.g., knee, hip, and shoulder replacement surgery. Trauma may also cause acute pain. Representative examples of traumatic events include sports-related injuries, military/combat injuries, automobile accidents and childbirth, wherein the time of injury can be precisely delineated.

Further provided is a method (Method 2) of reduction in Time to Recovery defined as time to loss of clinically important pain (<3 on a 0-10 NRS) in a patient in need thereof wherein the method comprises administering to a patient, following the onset of acute pain or initiation of a stimulus that causes acute pain such as surgery or trauma, amitifadine in a dose sufficient to inhibit or prevent the acute to subacute pain transition in the patient following the onset of acute pain or initiation of a stimulus that causes acute pain such as surgery or trauma compared to a placebo (i.e., a patient not receiving amitifadine in a comparable presentation).

Provided is a method (Method 3) of inhibiting or preventing the acute to chronic pain transition defined as a reduction or prevention in persistent pain (e.g., PPSP) or chronic pain intensity (Clinically Important PPSP) on a 0-10 point numerical rating scale (NRS) and associated pain symptoms including allodynia, aversion, and/or hyperalgesia at 12- or 24-weeks following the onset of acute pain or initiation of a stimulus that causes acute pain such as surgery or trauma. The method comprises administering to a patient, following the onset of acute pain or initiation of a stimulus that causes acute pain such as surgery or trauma, amitifadine in a dose sufficient to inhibit or prevent the acute to chronic transition in the patient (e.g., a post-surgical patient) compared to a placebo (i.e., a patient not receiving amitifadine in a comparable presentation).

Provided is a method (Method 4) of inhibiting or preventing the acute to chronic pain transition defined as a reduction or prevention in persistent pain (e.g., PPSP) or chronic pain intensity (Clinically Important PPSP) on a 0-10 point numerical rating scale (NRS) and associated pain symptoms including allodynia, aversion, and/or hyperalgesia, as well as associated affective and executive function symptoms including anhedonia, anxiety, catastrophizing, cognitive loss, depression, dysphoria, increase in impulsivity, and loss of motivation at 12- or 24-weeks following the onset of acute pain or initiation of a stimulus that causes acute pain such as surgery or trauma. The method comprises administering to a patient, following the onset of acute pain or initiation of a stimulus that causes acute pain such as surgery or trauma, amitifadine in a dose sufficient to inhibit or prevent the acute to chronic transition in the patient following surgery compared to a placebo (i.e., a patient not receiving amitifadine in a comparable presentation).

Provided is a method (Method 5) of inhibiting or preventing the acute to chronic pain transition defined as a reduction or prevention in persistent pain (e.g., PPSP) or chronic pain intensity (Clinically Important PPSP) on a 0-10 point numerical rating scale (NRS) and associated pain symptoms including allodynia, aversion, and/or hyperalgesia, as well as associated affective and executive function symptoms including anhedonia, anxiety, catastrophizing, cognitive loss, depression, dysphoria, increase in impulsivity, and motivation, using a composition with low risk of abuse, at 12- or 24-weeks following the onset of acute pain or initiation of a stimulus that causes acute pain such as surgery or trauma. The method comprises administering to a patient, following the onset of acute pain or initiation of a stimulus that causes acute pain such as surgery or trauma, amitifadine in a dose sufficient to inhibit or prevent the acute to chronic transition in the patient (e.g., a post-surgical patient) compared to a placebo (i.e., a patient not receiving amitifadine in a comparable presentation).

Provided is a method (Method 6) of inhibiting or preventing the acute to chronic pain transition defined as a reduction or prevention in persistent pain (e.g., PPSP) or chronic pain intensity 3 (Clinically Important PPSP) on a 0-10 point numerical rating scale (NRS) and hypodopaminergia-associated pain symptoms including allodynia, aversion, and/or hyperalgesia at 12- or 24-weeks following the onset of acute pain or initiation of a stimulus that causes acute pain such as surgery or trauma. The method comprises administering to a patient, following the onset of acute pain or initiation of a stimulus that causes acute pain such as surgery or trauma, amitifadine in a dose sufficient to inhibit or prevent the acute to chronic transition in the patient (e.g., a post-surgical patient) compared to a placebo (i.e., a patient not receiving amitifadine in a comparable presentation).

Provided is a method (Method 7) of inhibiting preventing the acute to chronic pain transition defined as a reduction or prevention in persistent pain (e.g., PPSP) or chronic pain intensity 3 (Clinically Important PPSP) on a 0-10 point numerical rating scale (NRS) and hypodopaminergia-associated pain symptoms including allodynia, aversion, and/or hyperalgesia, as well as associated affective and executive function symptoms including anhedonia, anxiety, catastrophizing, cognitive loss, depression, dysphoria, increase in impulsivity, and loss of motivation at 12- or 24-weeks following the onset of acute pain or initiation of a stimulus that causes acute pain such as surgery or trauma. The method comprises administering to a patient, following the onset of acute pain or initiation of a stimulus that causes acute pain such as surgery or trauma, amitifadine in a dose sufficient to inhibit or prevent the acute to chronic transition in the patient (i.e., a post-surgical patient) compared to a placebo (i.e., a patient not receiving amitifadine in a comparable presentation).

Provided is a method (Method 8) of inhibiting or preventing the acute to chronic pain transition defined as a reduction or prevention in persistent pain (e.g., PPSP) or chronic pain intensity 3 (Clinically Important PPSP) on a 0-10 point numerical rating scale (NRS) and hypodopaminergia-associated pain symptoms including allodynia, aversion, and/or hyperalgesia, as well as associated affective and executive function symptoms including anhedonia, anxiety, catastrophizing, cognitive loss, depression, dysphoria, increase in impulsivity, and loss of motivation, using a composition with low risk of abuse, at 6-months following the onset of acute pain or initiation of a stimulus that causes acute pain such as surgery or trauma. The method comprises administering to a patient, following the onset of acute pain or initiation of a stimulus that causes acute pain such as surgery or trauma, amitifadine in a dose sufficient to provide a reduction or prevention of the acute to chronic transition in the patient (e.g., a post-surgical patient) compared to a placebo (i.e., a patient not receiving amitifadine in a comparable presentation).

Provided is a method (Method 9) of inhibiting or preventing the acute to chronic pain transition defined as a reduction or prevention in persistent post-surgical pain (PPSP) or chronic pain intensity ≥3(Clinically Important PPSP) on a 0-10 point numerical rating scale (NRS) or reduction in Time to Recovery defined as time to loss of clinically important post-surgical pain (<3 on a 0-10 NRS) and hypodopaminergia-associated pain symptoms including allodynia, aversion, and/or hyperalgesia, as well as associated affective and executive function symptoms including anhedonia, anxiety, cognitive loss, depression, dysphoria, increase in impulsivity, and loss of motivation, in a female patient (e.g., adult women). This method applies to both the acute to subacute pain transition period of week 0 through week 12 (subacute period) following the onset of acute pain or initiation of a stimulus that causes acute pain such as surgery or trauma or in the acute to chronic pain transition period of week 4 through week 12 or later following the onset of acute pain or initiation of a stimulus that causes acute pain such as surgery or trauma, in a female patient in need thereof. The method comprises administering to a female patient, following the onset of acute pain or initiation of a stimulus that causes acute pain such as surgery or trauma, amitifadine in a dose sufficient to inhibit or prevent the acute to subacute or acute to chronic pain transition in the female patient (e.g., a post-surgical female patient) compared to a placebo (i.e., a female patient not receiving amitifadine in a comparable presentation).

Provided is a method (Method 10) of reducing or preventing cortical gray matter loss as a result of the acute to chronic pain transition defined as improvements from baseline in functional measurements of cortical regions of interest. A non-limiting example is use of functional MRI to measure cortical thickness in the left dorsolateral Prefrontal Cortex (DLPFC) from baseline to endpoint. This functional measurement would be in addition to evaluation of persistent post-surgical pain (PPSP) or chronic pain intensity (Clinically Important PPSP) on a 0-10 point numerical rating scale (NRS) and hypodopaminergia-associated pain symptoms including allodynia, aversion, and/or hyperalgesia, as well as associated affective and executive function symptoms including anhedonia, anxiety, catastrophizing, cognitive loss, depression, dysphoria, increase in impulsivity, and loss of motivation, using a composition with low risk of abuse, at 12- or 24-weeks post-surgery in a patient in need thereof. The method comprises administering to a patient, following the onset of acute pain or initiation of a stimulus that causes acute pain such as surgery or trauma, amitifadine in a dose sufficient to inhibit or prevent the acute to chronic transition in the patient (e.g., a post-surgical patient) compared to a placebo (i.e., a patient not receiving amitifadine in a comparable presentation).

Provided is a method (Method 11) of inhibiting or preventing the acute to chronic pain transition defined as a reduction or prevention in persistent pain (e.g., PPSP) or chronic pain intensity 3 (Clinically Important PPSP) on a 0-10 point numerical rating scale (NRS) and hypodopaminergia-associated pain symptoms including allodynia, aversion, and/or hyperalgesia, as well as associated affective and executive function symptoms including anhedonia, anxiety, catastrophizing, cognitive loss, depression, dysphoria, increase in impulsivity, and loss of motivation at 12- or 24-weeks following the onset of acute pain or initiation of a stimulus that causes acute pain such as surgery or trauma. The method comprises administering to a patient, following the onset of acute pain or initiation of a stimulus that causes acute pain such as surgery or trauma, amitifadine in a dose sufficient to inhibit or prevent the acute to chronic transition in a patient (e.g., a post-surgical patient) compared to a patient not receiving amitifadine in a comparable presentation.

Provided is a method (Method 12) to therapeutically address the critical time period in pain transition from acute pain to subacute pain which is between day 0 to 4 weeks and from week 4 to week 12 from the acute onset of pain. Amitifadine is administered to a patient between day 0 to week 12 from the acute onset of pain and in a dose sufficient to inhibit or prevent the acute to subacute transition in a patient following the onset of acute pain or initiation of a stimulus that causes acute pain, e.g., surgery, compared to a patient not receiving amitifadine in a comparable presentation. In one embodiment, an effective dose of amitifadine is administered to a patient over a period of at least 4 weeks. For example, amitifadine may begin upon initiation of a painful stimulus, e.g., emergency surgery, and then continue for another 4 weeks.

In another embodiment, an effective dose of amitifadine is administered to a patient over a period of at least 6 weeks. For example, amitifadine may begin 2 weeks prior to the initiation of a painful stimulus, e.g., surgery and then continue for another 4 weeks.

In another embodiment, an effective dose of amitifadine is administered to a patient over a period of at least 14 weeks. For example, amitifadine may begin 2 weeks prior to the initiation of a painful stimulus, e.g., surgery and then continue for another 12 weeks.

Provided is a method (Method 13) to therapeutically address the critical time period in pain transition from acute pain to chronic pain which is between day 0 to weeks 12 or more from the acute onset of pain or the initiation of a stimulus that causes acute pain. Amitifadine is administered to a patient in a dose sufficient to inhibit or prevent the acute to chronic transition in a patient following the acute onset of pain or the initiation of a stimulus that causes acute pain, e.g., surgery, compared to a patient not receiving amitifadine in a comparable presentation. In one embodiment, an effective dose of amitifadine is administered to a patient through week 12, or typically week 24. For example, amitifadine may begin 2 weeks prior to the initiation of a painful stimulus, e.g., surgery and then continue for another 24 weeks.

In another embodiment, an effective dose of amitifadine is administered to a patient over a period of at least 37 weeks. For example, amitifadine may begin 1 week prior to the initiation of a painful stimulus, e.g., surgery and then continue for another 36 weeks.

In another embodiment, an effective dose of amitifadine is administered to a patient over a period of at least 49 weeks. For example, amitifadine may begin 1 week prior to the initiation of a painful stimulus, e.g., surgery and then continue for another 48 weeks.

In another embodiment, an effective dose of amitifadine is administered over a period of at least 53 weeks. For example, amitifadine may begin 1 week prior to the initiation of a painful stimulus, e.g., surgery and then continue for another 52 weeks.

In one embodiment, an effective dose of amitifadine is administered to a patient beginning one week prior to initiation of a painful stimulus, e.g., a surgery and then continue for a period of one, three, or fifty-one weeks, or greater. In another embodiment, the effective dose could have been initiated two weeks prior to initiation of a painful stimulus, e.g., a surgery and then continue for a given period.

Provided is a method (Method 14) of reducing or preventing the acute to chronic pain transition defined as a reduction or prevention in persistent pain (e.g., PPSP) or chronic pain intensity (Clinically Important PPSP) on a 0-10 point numerical rating scale (NRS) or reduction in Time to Recovery defined as time to loss of clinically important post-surgical pain (<3 on a 0-10 NRS) and hypodopaminergia-associated pain symptoms including allodynia, aversion, and/or hyperalgesia, as well as associated affective and executive function symptoms including anhedonia, anxiety, catastrophizing, cognitive loss, depression, dysphoria, increase in impulsivity, and loss of motivation, particularly in a patient with pain catastrophization. This method would apply to both the acute to subacute pain transition period 0-4 weeks and 4-12 weeks post-surgery or in the acute to chronic pain transition period 0-12 weeks or more post-surgery, in a male or female patient in need thereof wherein the method comprises administering to a patient, amitifadine in a dose sufficient to provide a reduction or prevention of the acute to subacute or acute to chronic pain transition in a patient following surgery compared to a patient not receiving amitifadine in a comparable presentation.

Provided is a method (Method 15) for the use of the multimodal compound amitifadine to unexpectedly inhibit, prevent, alleviate, moderate, or reverse the acute to subacute pain transition or acute to chronic pain transition effected by modulation of multiple monoamine neurotransmitters or biogenic amines in different ratios resulting from multiple pharmacology and multiple sites of activity. This method pertains to the unanticipated effects of a medication combining (1) known pain/mood effects of 5-HT/NE reuptake inhibition plus; (2) with the unappreciated pain effects on the DA reuptake inhibition on descending pathways; plus (3) 5-HT2c partial agonism descending pathway pain effects; plus (4) prophetic 5-HT2c partial agonist effects on excitatory glutamatergic pyramidal neurons in the PFC; plus (5) DA reuptake inhibitory activity to correct hypodopaminergia; with (6) protection from abuse (due to 5-HT2c partial agonism effects at the NAc). The multimodal aspects of amitifadine, particularly interaction effects among the different systems to yield this mix of pharmacology makes the potential to anticipate prevention of chronic pain possible. Although a portion of this is known, e.g., 5-HT/NE reuptake inhibitory effects on pain in the descending pathways, on the other hand, DA reuptake inhibitor effects on DA dependent descending pain pathways is not widely appreciated. Neither are 5-HT2c partial agonist effects on VLO descending pain pathways. Serotonin 2c partial agonist effects on excitatory glutamatergic pyramidal neurons in the PFC to reduce allodynia, pain aversion, and affective symptoms are not appreciated. Furthermore, amitifadine effects on pain-induced hypodopaminergia are not well recognized, thus, bringing all of these disparate effects together to inhibit or prevent the acute to subacute or acute to chronic pain transition through effects on pain and emotional/cognitive symptoms have heretofore not been recognized. With pharmacology affecting multiple brain regions (NAc, VAT, PFC [including PFC subregions DLPFC, mPFC [further divided into prelimbic cortex (PL-PFC) and infralimbic cortex (IL-PFC)], and VLO and with unanticipated outcomes, e.g., 5-HT2c partial agonist inhibition causing a reduction in phasic DA release at NAc yet with the potential for glutamatergic effects at PFC, all of which inhibit aversion and allodynia at NAc, plus direct anti-nociceptive effects in the VLO, it is highly unlikely that a prevention in the acute to sub/chronic pain transition could be prospectively anticipated. Further unexpected utility is anticipated because of multimodal pharmacology imparted by multiple ratios. Specifically, amitifadine inhibits the serotonin transporter at 12 nM, the norepinephrine transporter at 23 nM, and the dopamine transporter at 96 nM. Amitifadine simultaneously has partial agonism effects at mesocorticolimbic 5-HT2c receptors at 47 nM. This multimodal activity is predicted to inhibit or prevent the acute to subacute and acute to chronic pain transition, and the allodynia, aversion, and hyperalgesia, and the affective and executive function symptoms associated with the acute to chronic pain transition in both males, and especially so in females. Amitifadine multimodal activity is also predicted to reverse structural brain changes with corresponding functional improvement in male and especially so, in female patients with preexisting chronic pain. These hypodopaminergia-associated symptoms of chronic pain include pain aversion, allodynia, and hyperalgesia as well as the affective symptoms of pain including anhedonia, anxiety, depression, cognitive loss, dysphoria, increase in impulsivity, and loss of motivation. Amitifadine enables the above while maintaining a low risk of abuse liability.

Provided is a method (Method 16) for the use of multimodal compound, amitifadine that may have different ratios of monoamine reuptake inhibition to optimize pharmacological properties. A focus of this method is the ratio of reuptake inhibitory activity for each of the three transporters: SERT, NET, and DAT and importantly, between the three, i.e., the relationship among the three is critically important, especially how ICₛo for DA reuptake inhibition (96 nM) relates to SERT (12 nM) and NET (23 nM). Specifically, amitifadine has a relationship between reuptake inhibition at the 5-HT and NE transporters between approximately 5 to 10 times the potency of the DA transporter inhibition. This is predicted to be the optimal relationship of pain/mood/cognition efficacy vs. tolerability for monoamine reuptake inhibitors. The preferred potency of each transporter has been an on-going argument for several decades. Amitifadine teaches away from the accepted transporter occupancy dogma at each transporter of greater than 75% occupancy at SERT (Meyer J, et al. Serotonin transporter occupancy of five selective serotonin reuptake inhibitors at different doses: an^{[11c]}DASB positron emission tomography study. Am J Psychiatry. 2004 May; 161(5):826-35.) and 25% at DAT (Learned-Coughlin S, et al. In vivo activity of bupropion at the human dopamine transporter as measured by positron emission tomography. Biol Psychiatry. 2003 Oct 15;54(8):800-5.). The triple reuptake inhibitor field is littered with failures, in no small part due to the unpredictability with respect to the optimal ratio of the three transporters. The 5-HT2c activity adds yet another dimension of unpredictability. The dose adds a fifth element, i.e., at low doses little dopaminergic effect is observed whereas a higher effect is observed at higher doses. In the case of amitifadine, this multimodal compound has an unbalanced serotonin—norepinephrine—dopamine reuptake inhibition ratio of^{—}1:2:8, respectively, combined with 5-HT2c partial agonism that allows for higher dosages of amitifadine, (+)-1-(3,4-dichlorophenyI)-3-azabicyclo[3.1.0]hexane to be used without triggering the poor tolerability from dopaminergic (e.g., high rates of insomnia, euphoric mood) or norepinephrine side effects (e.g., nausea, excessively elevated heart rate/blood pressure) observed with balanced triple reuptake inhibitors or unbalanced triple reuptake inhibitors with markedly different inhibition ratios. These preliminary data suggest that triple reuptake inhibition may still provide effective prevention and treatment for pain when the relative inhibitory potencies are optimal, and the compound is well tolerated. The appropriate ratio of inhibitory potencies, especially potency for the DA transporter relative to 5-HT and NE transporters to optimize efficacy while maintaining tolerability has heretofore been difficult to achieve and development failure in the triple field has been the norm. Adding a relevant fourth pharmacology to the triple reuptake inhibitor adds an order of magnitude of unpredictability as to assessing whether a given set of reuptake ratios or combination of pharmacology is safe and effective at a given set of doses.

Provided is a method (Method 17) of reducing or preventing the acute to chronic pain transition defined as a reduction or prevention in persistent post-surgical pain (PPSP) or chronic pain intensity (Clinically Important PPSP) on a 0-10 point numerical rating scale (NRS) or reduction in Time to Recovery defined as time to loss of clinically important post-surgical pain (<3 on a 0-10 NRS) and hypodopaminergia-associated pain symptoms including allodynia, aversion, and/or hyperalgesia, as well as associated affective and executive function symptoms including anhedonia, anxiety, catastrophizing, cognitive loss, depression, dysphoria, increase in impulsivity, and loss of motivation, particularly in a high pain catastrophizing patient. This could be a pre-operative breast surgery or total knee surgery patient or a patient anticipating an exacerbation of worsening pain with the following characteristic symptoms of pain catastrophization: , e.g., a person: (1) who ruminates about the impending pain, (2) who magnifies the seriousness of the pain, and (3) yet feels helpless about the pain. The level of pain catastrophization may be measured by the Pain Catastrophization Scale (PCS) and a score of >16 is considered high. This method would apply to both the acute to subacute pain transition period 0-4 weeks and 4-12 weeks post-surgery or in the acute to chronic pain transition period 0 to week 12 or more post-surgery, in a male or female patient in need thereof wherein the method comprises administering to a patient, amitifadine in a dose sufficient to provide a reduction or prevention of the acute to subacute or acute to chronic pain transition in a patient following surgery compared to a patient not receiving amitifadine in a comparable presentation.

Provided is a method (Method 18) of reducing or preventing the acute to chronic pain transition defined as a reduction or prevention in persistent post-surgical pain (PPSP) or chronic pain intensity (Clinically Important PPSP) on a 0-10 point numerical rating scale (NRS) or reduction in Time to Recovery defined as time to loss of clinically important post-surgical pain (<3 on a 0-10 NRS) and hypodopaminergia-associated pain symptoms including allodynia, aversion, and/or hyperalgesia, as well as associated affective and executive function symptoms including anhedonia, anxiety, catastrophizing, cognitive loss, depression, dysphoria, increase in impulsivity, and loss of motivation, particularly in a patient requiring soft-tissue surgery, for example breast cancer surgery including breast-conserving surgery (lumpectomy) and/or mastectomy. Other examples could include but are not limited to surgeries to repair a hernia or cholecystectomy. This method would apply to both the acute to subacute pain transition period 0-4 weeks and 4-12 weeks post-surgery or in the acute to chronic pain transition period 0 to week 12 or more post-surgery, in a male or female patient in need thereof wherein the method comprises administering to a patient, amitifadine in a dose sufficient to provide a reduction or prevention of the acute to subacute or acute to chronic pain transition in a patient following surgery compared to a patient not receiving amitifadine in a comparable presentation.

Provided is a method (Method 19) of reducing or preventing the acute to chronic pain transition defined as a reduction or prevention in persistent post-surgical pain (PPSP) or chronic pain intensity (Clinically Important PPSP) on a 0-10 point numerical rating scale (NRS) or reduction in Time to Recovery defined as time to loss of clinically important post-surgical pain (<3 on a 0-10 NRS) and hypodopaminergia-associated pain symptoms including allodynia, aversion, and/or hyperalgesia, as well as associated affective and executive function symptoms including anhedonia, anxiety, catastrophizing, cognitive loss, depression, dysphoria, increase in impulsivity, and loss of motivation, particularly in a patient requiring orthopedic surgery, for example total knee replacement or total hip replacement. This method would apply to both the acute to subacute pain transition period 0-4 weeks and 4-12 weeks post-surgery or in the acute to chronic pain transition period 0 to week 12 or more post-surgery, in a male or female patient in need thereof wherein the method comprises administering to a patient, amitifadine in a dose sufficient to provide a reduction or prevention of the acute to subacute or acute to chronic pain transition in a patient following surgery compared to a patient not receiving amitifadine in a comparable presentation.

Other aspects of the present disclosure provide methods of treating chronic pain that entail administering a therapeutically effective dose of amitifadine to a patient suffering from chronic pain. In addition to reducing the level of chronic pain, the present methods may also be effective to ameliorate one or more types of hypodopaminergia-associated symptoms of chronic pain including pain aversion, allodynia, and/or hyperalgesia, as well as affective symptoms of chronic pain that may include anhedonia, anxiety, catastrophizing, cognitive loss, dysphoria, increase in impulsivity, and loss of motivation. In some embodiments, the patient has chronic lower back pain (CLBP). In some embodiments, the patient is female. Symptomatic improvements may also be reflected in structural improvements in a target region of interest (ROI), e.g., the left dorsolateral prefrontal cortex (DLPFC) as evidenced by increased cortical thickening and improvement of cognitive task-related brain activity. In some embodiments, the treatment regimen may last upwards of 24 weeks, 52 weeks or longer.

In some embodiments, the method (Method 20) is effective to reverse structural brain changes with corresponding functional improvement in patients with preexisting chronic pain. The method comprises administering to the patient amitifadine to effect treatment of the hypodopaminergia-associated symptoms of chronic pain including allodynia, aversion, and/or hyperalgesia, as well as associated affective and executive function symptoms including anhedonia, anxiety, catastrophizing, cognitive loss, depression, dysphoria, increase in impulsivity, and loss of motivation that the patient may be experiencing. In some embodiments, a patient with chronic lower back pain (CLBP) who is effectively treated with amitifadine may experience reduced symptoms of chronic pain including less pain aversion, less allodynia, and less hyperalgesia and improvements on affective symptoms of pain including anhedonia, anxiety, catastrophizing, cognition, depression, dysphoria, impulsivity, and motivation resulting in structural improvements in an expected target region of interest (ROI), e.g., the left dorsolateral prefrontal cortex (DLPFC) as evidenced by increased cortical thickening and improvement in cognitive task-related brain activity.

In some embodiments, the method (Method 21) is effective to reverse structural brain changes with corresponding functional improvement in patients with preexisting chronic pain. The method comprises administering to the patient amitifadine to effect treatment of the hypodopaminergia-associated symptoms of chronic pain including allodynia, aversion, and/or hyperalgesia, as well as associated affective and executive function symptoms including anhedonia, anxiety, catastrophizing, cognitive loss, depression, dysphoria, increase in impulsivity, and loss of motivation that the patient may be experiencing. In some embodiments, a patient with chronic lower back pain (CLBP) who is effectively treated with amitifadine may experience reduced symptoms of chronic pain including less pain aversion, less allodynia, and less hyperalgesia and improvements on affective symptoms of pain including anhedonia, anxiety, catastrophizing, cognition, depression, dysphoria, impulsivity, and motivation resulting in structural improvements in an expected target region of interest (ROI), e.g., the left dorsolateral prefrontal cortex (DLPFC) as evidenced by increased cortical thickening and improvement of cognitive task-related brain activity. In another embodiment, administering an effective dose of amitifadine to a patient in need thereof during a period of at least 24 weeks to reduce or eliminate chronic pain and affective symptoms as well as physical measures of brain function following diagnosis of pre-existing chronic pain. In another embodiment, administering an effective dose of amitifadine to a patient in need thereof during a period of at least 52 weeks ameliorates chronic pain, affective symptoms, executive function symptoms, and quality of life (QoL) measures such as EQ-5D following diagnosis of pre-existing chronic pain.

In some embodiments, the method (Method 22) is effective to reverse structural brain changes with corresponding functional improvement in a female patient with preexisting chronic pain. The method comprises administering to the female patient an effective dose of amitifadine to treat the hypodopaminergia-associated symptoms of chronic pain and associated pain symptoms including allodynia, aversion, and/or hyperalgesia, as well as associated affective and executive function symptoms including anhedonia, anxiety, cognitive loss, depression, dysphoria, increase in impulsivity, and loss of motivation. In some embodiments, the female patient has chronic lower back pain (CLBP) and may experience reduced pain and reduced affective symptoms which results in structural changes in an expected target ROI, e.g., the left dorsolateral prefrontal cortex (DLPFC) with increased cortical thickening demonstrating improvement of cognitive task-related brain activity. This method would apply to both the acute to subacute pain transition period 0-4 and 4-12 weeks post-CLBP exacerbation (initiating event) or in the acute to chronic pain transition period 0 to week 12 or more post-CLBP exacerbation (initiating event).

Provided is a method (Method 23) treatment with amitifadine and reducing or preventing the acute to chronic pain transition defined as a reduction or prevention in persistent post-surgical pain (PPSP) or chronic pain intensity (Clinically Important PPSP) on a 0-10 point numerical rating scale (NRS) or reduction in Time to Recovery defined as time to loss of clinically important post-surgical pain (<3 on a 0-10 NRS) and reducing exposure to potentially injurious pain medications such as opioids during the initial 0 to 12 months following initiation of therapy compared to those not receiving amitifadine.

Provided is a method (Method 24) treatment with amitifadine and reducing or preventing the acute to subacute pain transition defined as a reduction or prevention in persistent post-surgical pain (PSP) or reduction in Time to Recovery defined as time to loss of clinically important post-surgical pain (<3 on a 0-10 NRS) and reducing exposure to potentially injurious pain medications such as opioids during the initial 0-4 weeks and 4-12 weeks following initiation of therapy compared to those not receiving amitifadine.

Accordingly, the disclosure provides a method of prophylaxis or inhibiting the development of chronic pain in a patient in need thereof, comprising administering an effective amount of a novel multimodal composition, e.g., amitifadine, in free base or pharmaceutically acceptable salt form, during a period of at least 12 weeks following the initiation of a painful stimulus, e.g., surgery.

Further provided with respect to any of Methods 1-24 as follows:
1.1 Any of Method 1-24, wherein (1R,5S)-1-(3,4-dichlorophenyl)-3-azabicyclo[3.1.0]hexane is in pharmaceutically acceptable salt form;
1.2 Any Method 1-24 or 1.1, wherein (1R,5S)-1-(3,4-dichlorophenyl)-3-azabicyclo[3.1.0]hexane in pharmaceutically acceptable salt form is an acid addition salt;
1.3 Any of Method 1-24, 1.1, or 1.2, wherein (1R,5S)-1-(3,4-dichlorophenyl)-3-azabicyclo[3.1.0)hexane in pharmaceutically acceptable salt form is (1R,5S)-1-(3,4-dichlorophenyl)-3-azabicyclo[3.1.0] hexane hydrochloride;
1.4 Any of Method 1-24 or 1.1-1.3, wherein (1R,5S)-1-(3,4-dichlorophenyl)-3-azabicyclo[3.1.0]hexane in pharmaceutically acceptable salt form is crystalline;
1.5 Any of Method 1-24 or 1.1-1.4, wherein (1R,5S)-1-(3,4-dichlorophenyl)-3-azabicyclo[3.1.0]hexane, in free or pharmaceutically acceptable salt form, is Polymorph A, for example, Polymorph A substantially free of other polymorphic forms;
1.6 Any of Method 1-24 or 1.1-1.4, wherein (1R,5S)-1-(3,4-dichlorophenyl)-3-azabicyclo[3.1.0]hexane, in free or pharmaceutically acceptable salt form, is Polymorph B, for example, Polymorph B substantially free of other polymorphic forms;
1.7 Any of Method 1-24 or 1.1-1.4, wherein (1R,5S)-1-(3,4-dichlorophenyl)-3-azabicyclo[3.1.0]hexane, in free or pharmaceutically acceptable salt form, is Polymorph C, for example, Polymorph C substantially free of other polymorphic forms;
1.8 Any of Method 1-24 or 1.1-1.7, wherein the method comprises administering (1R,5S)-1-(3,4-dichlorophenyl)-3-azabicyclo[3.1.0]hexane, in free or pharmaceutically acceptable salt form, once, twice, three, or four times daily, e.g., once daily, e.g., twice daily, e.g., three times daily, e.g. , four times daily;
1.9 Any of Method 1-24 or 1.1-1.8, wherein the method comprises administering 75 mg to 200 mg of (1R,5S)-1-(3,4-dichlorophenyl)-3-azabicyclo[3.1.0]hexane, in free or pharmaceutically acceptable salt form, per day, for example, comprising administering 100 mg to 200 mg of (1R,5S)-1-(3,4-dichlorophenyl)-3-azabicyclo[3.1.0]hexane, in free or pharmaceutically acceptable salt form, per day, for example, comprising administering 150 mg of (1R,5S)-1-(3,4-dichlorophenyl)-3-azabicyclo[3.1.0]hexane, in free or pharmaceutically acceptable salt form, per day (e.g., by administering 75 mg twice per day, e.g., by administering three 25 mg tablets in the morning and three 25 mg tablets in the afternoon);
1.10 Any of Method 1-24 or 1.1-1.9, wherein the pain is acute pain, e.g., acute post-surgical pain during weeks 0-4. For instance, any of Method 1-24 or 1.1-1.9, wherein the pain is post-breast surgery pain (e.g., post-lumpectomy/breast conservation surgery, or mastectomy). For instance, any of Method 1-24 or 1.1-1.9, wherein the pain is subacute during weeks 4-12. For instance, any of Method 1-24 or 1.1-1.9, wherein the pain is subacute: post-surgical breast-surgery, post-surgery thoracotomy, post-hip/knee surgery. For instance, any of Method 1-24 or 1.1-1.9, wherein the pain is chronic during weeks 12 or later. For instance, any of Method 1-24 or 1.1-1.9, wherein the pain is chronic: post-surgical breast-surgery, post-surgery thoracotomy, post-hip/knee surgery;

Any foregoing method wherein the novel multimodal composition, e.g., amitifadine is administered over a period of about 0-4 weeks and 4-12 weeks from the initial painful stimulus in weekly intervals to address the acute to subacute transition period or up to 0 to 12 weeks or later in weekly intervals to cover the acute to chronic transition period following the initiation of the painful stimulus;

Any foregoing method, wherein the painful stimulus is surgery including but not limited to amputation, breast-surgery (lumpectomy/breast-conserving surgery/mastectomy), cardiac surgery especially involving sternotomy, cholecystectomy, hernia repair, hip/knee surgery, and thoracotomy;

Any foregoing method, wherein the subacute and chronic pain condition is persistent post-surgical pain (PPSP);

Any foregoing method wherein the painful stimulus is a traumatic injury, e.g., sports-related injuries, military/combat injuries, automobile accidents and childbirth or a neck, hip injury, or back injury;

Any foregoing method wherein the painful stimulus is surgery, and the administration of novel multimodal composition, e.g., amitifadine commences prior to the surgery, e.g., commences up to two weeks before the surgery, e.g., 14 days, seven days, three days or one day before the surgery;

Any foregoing method wherein the patient also receives analgesic medications in addition to novel multimodal composition, e.g., amitifadine, for example opioids, nerve blocks, anesthetics, and/or post-operative analgesics, e.g., acetaminophen. In some embodiments, the patient is treated with amitifadine without concomitant administration of an analgesic;

Any foregoing method wherein the daily dosage of novel multimodal composition, e.g., amitifadine is 75 mg to 300 mg, e.g., about 150 mg, e.g., 75 mg b.i.d., e.g., once in the morning and once in the evening;

Any foregoing method wherein the daily dosage of novel multimodal composition, e.g., amitifadine is tapered off at the end of the administration period, e.g., from 150 mg daily (75 mg b.i.d.) to 75 mg daily (e.g., 75 mg once daily), over a period of 3-7 days;

Any foregoing method wherein the administration of novel multimodal composition, e.g., amitifadine is effective to increase tonic dopamine neurotransmission in the mesolimbic area, thereby inhibiting or preventing the transition of acute pain to subacute pain;

Any foregoing method wherein the administration of novel multimodal composition, e.g., amitifadine is effective to increase tonic dopamine neurotransmission in the mesolimbic area, thereby inhibiting or preventing the transition of acute pain to chronic pain;

Any foregoing method wherein the administration of novel multimodal composition, e.g., amitifadine is effective to modulate glutamate neurotransmission from the PFC, thereby inhibiting or preventing the transition of acute pain to subacute pain;

Any foregoing method wherein the administration of novel multimodal composition, e.g., amitifadine is effective to modulate glutamate neurotransmission from the PFC, thereby inhibiting or preventing the transition of acute pain to chronic pain;

Any foregoing method wherein the administration of novel multimodal composition, e.g., amitifadine is effective to decrease phasic dopamine neurotransmission from the NAc, thereby inhibiting or preventing the transition of acute pain to subacute pain;

Any foregoing method wherein the administration of novel multimodal composition, e.g., amitifadine is effective to decrease phasic dopamine neurotransmission from the NAc, thereby inhibiting or preventing the transition of acute pain to chronic pain;

Any foregoing method wherein the administration of novel multimodal composition, e.g., amitifadine is effective to decrease phasic dopamine neurotransmission from the mesolimbic area (e.g., NAc), thereby decreasing the potential for drug abuse liability;

Any foregoing method wherein the administration of novel multimodal composition, e.g., amitifadine is effective to increase tonic dopamine neurotransmission in the mesolimbic area, thereby inhibiting or preventing the transition of acute pain to chronic pain as evidenced by reduction or prevention in persistent post-surgical pain (PPSP) or chronic pain intensity (Clinically Important PPSP) on a 0-10 point numerical rating scale (NRS) at 6-months post-surgery;

Any foregoing method wherein the administration of novel multimodal composition, e.g., amitifadine is effective to increase dopamine neurotransmission in the mesolimbic area, thereby inhibiting or preventing the transition of acute pain to chronic pain as evidenced by reduction in Time to Recovery defined as time to loss of clinically important post-surgical pain (<3 on a 0-10 NRS);

Any foregoing method wherein the administration of novel multimodal composition, e.g., amitifadine is effective to reduce the characteristic symptoms of the pain transition including allodynia (pain due to a stimulus that does not normally provoke pain), pain aversion (the unpleasantness of pain), and hyperalgesia (increased pain from a stimulus that normally provokes pain);

Any foregoing method wherein the administration of novel multimodal composition, e.g., amitifadine is effective to reduce anhedonia (inability to perceive pleasure), affective symptoms of pain including anxiety, depression, and dysphoria, and executive dysfunction including cognitive deficits, increase in impulsivity, and loss of motivation;

Any foregoing method wherein the patient has no prior history of chronic pain;

Any foregoing method wherein the patient suffers from anxiety;

Any foregoing method wherein the patient suffers from depression;

Any foregoing method wherein the administration of amitifadine in the pre-operative setting is effective to reduce catastrophizing as measured by a validated instrument, e.g., the Pain Catastrophizing Scale (PCS), thereby inhibiting or reducing the transition of acute pain to subacute pain and of acute pain to chronic pain;

Any foregoing method wherein the administration of amitifadine is effective to reduce impulsivity as measured by a validated instrument, e.g., Barratt Impulsiveness scale;

Any foregoing method wherein the peri-operative administration of amitifadine is effective to reduce the perioperative requirement of opioid pain medications;

Any foregoing method wherein the administration of amitifadine is effective to reduce the requirement of opioid pain medications during the subacute period;

Any foregoing method wherein the administration of amitifadine is effective in reversing the structural and functional deficits caused by chronic pain;

Any foregoing method wherein the administration of amitifadine is effective in reversing the structural and functional deficits caused by chronic pain;

Any foregoing method wherein the administration of amitifadine is effective in women coordinately treated with radiation therapy following breast surgery compared to women not treated with amitifadine;

Any foregoing method wherein the administration of amitifadine is effective in women coordinately treated with chemotherapy following breast surgery compared to women not treated with amitifadine;

and

Any foregoing method wherein the administration amitifadine is effective to increase mesolimbic dopamine in mammals experiencing pain yielding mesolimbic hypodopaminergia.

### Modes Of Administration Of Amitifadine, Formulations And Dosage Amounts

The mode of administration of amitifadine is not limited. (+)-1-(3,4-dichlorophenyl)-3-azabicyclo[3.1.0]hexane, in free or pharmaceutically acceptable salt form, may be administered by any suitable, medically acceptable route, including orally, parenterally, transdermally, inhalation, slow release, controlled release, although various other known delivery routes, devices and methods can likewise be employed. In some embodiments, amitifadine is administered via an oral controlled-release pharmaceutical composition comprising (+)-1-(3,4-dichlorophenyl)-3-azabicyclo[3.1.0]hexane, in free or pharmaceutically acceptable salt form. In some embodiments, provided is an oral immediate release pharmaceutical composition comprising (+)-1-(3,4-dichlorophenyl)-3-azabicyclo[3.1.0]hexane, in free or pharmaceutically acceptable salt form.

Pharmaceutical compositions comprising (+)-1-(3,4-dichlorophenyI)-3-azabicyclo[3.1.0]hexane, in free or pharmaceutically acceptable salt form, may be prepared using conventional diluents or excipients and techniques known in the galenic art. Thus, oral dosage forms may include tablets, capsules, solutions, suspensions, and the like. Parenteral dosage forms for perioperative or hospital use may be prepared using standard galenic techniques.

In other embodiments, amitifadine may be formulated for extended or controlled-release delivery. In one exemplary embodiment, amitifadine can be administered in a therapeutically effective amount within the methods of the disclosureby utilizing a dosage strength from about 10 mg to about 300 mg once or twice daily in an oral unit dosage composition containing the active ingredient. In some embodiments the maximum daily dose is 200 mg. In one exemplary embodiment, amitifadine may be administered in a therapeutically effective amount in up to a 1000 mg weight caplet-shaped tablet. using pharmaceutically suitable excipients including one or more controlled-release matrices, fillers, disintegrants, glidants, lubricants, and coatings. In one exemplary embodiment, amitifadine can be administered in a therapeutically effective amount within the methods of the disclosureby utilizing a dosage strength from about 8% to 14% by weight of the composition of a pharmaceutically acceptable carrier, e.g., a tablet with pharmaceutically suitable excipients such as fillers, disintegrants, glidants, lubricants, and coatings, and from about 15% to 45% by weight of the composition of a hydroxypropyl methyl cellulose controlled-release matrix. In other embodiments, amitifadine may be formulated using proprietary controlled-release technology to release at appropriate levels in the gastrointestinal tract while delivering therapeutically effective amount between 25 to 200 mg of amitifadine free base.

The following representative amitifadine formulations include two capsule formulations, both with immediate-release (IR) profiles, and 4 tablet formulations, two as an IR and two as a controlled-release (CR).

Amitifadine Size 3 Immediate-Release Capsule Formulation, 10 g/25 mg Capsules (standard)

| | | Wt/capsule | | Wt/capsule |
|---|---|---|---|---|
| Ingredient | Wt (%) | (mg) | Wt (%) | (mg) |
| Amitifadine HCl | 8.00 | 10.00 | 13.89 | 25.00 |
| Mannitol, USP (Pearlitol SD100); filler | 88.20 | 110.25 | 82.31 | 148.16 |
| Talc, USP; glidant | 3.00 | 3.75 | 3.00 | 5.40 |
| Magnesium stearate, NF⁽¹⁾; lubricant | 0.80 | 1.00 | 0.80 | 1.44 |
| Opaque white capsule (hard gelatin), Size 3 | | | | |
| Total | 100.00 | 125.00 | 100.00 | 180.00 |

⁽¹⁾NF, National Formulary

Amitifadine Size 3 Immediate-Release Capsule
Formulation, 10 g/25 mg Capsules (standard)

| | | Weight/ |
|---|---|---|
| Ingredient | Wt (%) | capsule (mg) |
| Amitifadine HCl | 8.93 | 25.00 |
| Mannitol, USP (Pearlitol SD100); filler | 87.27 | 244.36 |
| Talc, USP; glidant | 3.00 | 8.40 |
| Magnesium stearate, NF; lubricant | 0.80 | 2.44 |
| Swedish Orange (hard gelatin) Capsules, Size 1 | | |
| Total | 100.00 | 280.00 |

Amitifadine Immediate-Release Tablet Formulation⁽¹⁾

| Ingredient | Wt (%) | Wt (mg) |
|---|---|---|
| Amitifadine HCl | 12.5% | 25.000⁽²⁾ |
| Dibasic calcium phosphate, NF; Emcompress (filler) | 40.50% | 81.000⁽²⁾ |
| Microcrystalline cellulose, NF; Avicel (binder)⁽³⁾ | 40.50% | 81.50 |
| Croscarmellose sodium, NF; AcDiSol (disintegrant) | 5.00% | 10.00 |
| Colloidal Silicon Dioxide; CapOSil (glidant) | 0.75% | 1.50 |
| Magnesium Stearate, NF (lubricant) | 0.75% | 1.50 |
| Total | 100.0% | 200.00 |

⁽¹⁾Direct compression, white oval-shaped tablets
⁽²⁾API to be adjusted for purity and moisture; reduce the quantity of Emcompress to compensate
⁽³⁾MCC, Avicel PH102

Amitifadine Immediate-Release Tablet Formulation Coating

| Ingredient | Wt (%) | Mg/tablet |
|---|---|---|
| Amitifadine Core Tablet, 25 mg | | 200.00 |
| Opadry II White Film Coating (85F18422) | 3.00% | 6.00 |
| Purified water, USP⁽¹⁾ | | |
| Total | 103.0% | 206.00 |

⁽¹⁾Removed during the coating process. Not included in the Total column

Amitifadine Immediate-Release Tablet Formulation⁽¹⁾

| Ingredient | Wt (%) | Wt (mg) |
|---|---|---|
| Amitifadine HCl | 28.6% | 100.00 |
| HPMC (hydrophilic CR polymer)⁽²⁾ | 30.0% | 105.00 |
| MCC (disintegrant)⁽³⁾ | 20.4% | 71.5 |
| Starch 1500 pregelatinized starch (coating) | 20.0% | 70.00 |
| Colloidal Silicon Dioxide (glidant) | 0.5% | 1.75 |
| Magnesium Stearate (lubricant) | 0.5% | 1.75 |
| Total | 100.0% | 350.00 |

⁽¹⁾Direct compression, ⅜″ round, standard biconvex tablet
⁽²⁾HPMC, hydroxypropyl methylcellulose. Methocel K4 or K100
⁽³⁾MCC, microcrystalline cellulose, 90 micron grade

Amitifadine Immediate-Release Tablet Formulation⁽¹⁾

| Ingredient | Wt (%) | Wt (mg) |
|---|---|---|
| Amitifadine HCl | 30% | 150.00 |
| Hydroxypropylmethylcellulose (HPMC, | 30.0% | 150.00 |
| hydrophilic CR polymer)⁽²⁾ | | |
| Microcrystalline cellulose (MCC, | 20.0% | 100.00 |
| disintegrant)⁽³⁾ | | |
| Starch 1500 (pregelatinized starch, coating) | 19.0% | 95.00 |
| Colloidal Silicon Dioxide (glidant) | 0.5% | 2.50 |
| Magnesium Stearate (lubricant) | 0.5% | 2.50 |
| Total | 100.0% | 500.00 |

⁽¹⁾Direct compression, tablet configuration TBD
⁽²⁾HPMC, hydroxypropyl methylcellulose. Methocel K4 or K100
⁽³⁾MCC, microcrystalline cellulose, 90 micron grade

Clinical trials in pain use various instruments to assess pain, mood, anxiety, in addition to the Numeric Rating Scale (NRS).

Administration of pharmaceutical compositions comprising amitifadine in effective amounts are effective for inhibition of acute to chronic pain transition by improving an individual's score on one or more multidimensional scales. For inhibition of the acute to chronic transition in the chronic setting (e.g., subacute back pain transitioning to chronic lower back pain) those instruments include but are not limited to the Pain Sensitivity Questionnaire (PSQ), a 17-item instrument used to assess individual pain sensitivity; Pain Disability Index (PDI), an assessment of physical impairment in relation to pain; PainDETECT, a 12-item assessment of neuropathic-like symptoms. PDt includes questions of current pain intensity (Pain/c) and subjective report of average pain intensity over the past 4-weeks (Pain/4w); McGill Pain Questionnaire—Short Form (sf-MPQ), a validated measure assessing both sensory and affective components of pain (MPQ/s and MPQ/a). It also includes a visual analog scale (VAS) of pain; Pain Catastrophizing Scale (PCS), a 5-point instrument to assess thoughts or feelings on past pain experience. PCS yields three sub-scale scores assessing rumination (PCS/r), magnification (PCS/m), and helplessness (PCS/h); Beck Depression Inventory (BDI), a 21-item instrument for measuring the severity of depression; Positive and Negative Affect Scale (PANAS), has two mood scales, one measuring positive affect and the other measuring negative affect (PANAS/n). Each scale is rated on a 5-point, 10-item scale; DN4 ‘douleur neuropathique 4 questionnaire’ (i.e., neuropathic pain four questionnaire in French). The instrument is used for the diagnosis of neuropathic pain symptoms and signs associated with neuropathic pain and includes a series of four questions consisting of both sensory descriptors and signs related to a bedside sensory examination; Hospital Anxiety and Depression Scale (HADS) is comprised of two subscales, a depression subscale, and an anxiety subscale. The eight items composing the depression subscale were largely based on the anhedonic state since this is probably the central psychopathological feature of that form of depression which responds well to antidepressant drug treatment, and therefore provides the most useful information for the clinician. The eight items composing the anxiety subscale focus on the psychic manifestations of anxiety. Assessment of the overall severity of anxiety and depression were both rated on five-point (0-4) scales; Breast Cancer Pain Questionnaire (BCPQ) measures the prevalence of pain and sensory symptoms with dichotomous (yes/no) questions. Women are asked systematically to address 4 specific regions of symptoms: (1) area of the breast (defined as either the affected breast or the area from which the breast was removed), (2) the axilla, (3) the arm, and (4) the side of the body, rating pain severity and frequency in each region. Severity of pain is measured using an NRS 0-10 in which 0 indicated no pain and 10 indicated worst imaginable pain. For the reporting of results regarding severity of pain, scores of 1 to 3 were categorized as light pain, scores of 4 to 6 as moderate pain, and scores of 7 to 10 as severe pain. Worst pain was defined as the highest pain score of the 4 regional pain scores. The frequency of symptoms was assessed by a 3-point verbal categorical scale: (1) every day or almost every day, (2) 1 to 3 days a week, or (3) more rarely. Questions are asked about physician visits due to pain in the operated region, use of analgesics, other treatment for pain in the affected region, or all 3, and pain in other locations (e.g., low back pain, headache); and SF-36 questionnaire, a self-administered questionnaire containing 36 items which takes about five minutes to complete. It measures health on eight multi-item dimensions, covering functional status, well-being, and overall evaluation of health.

The disclosure is further illustrated in the following examples, which are meant to be exemplary and not limiting.

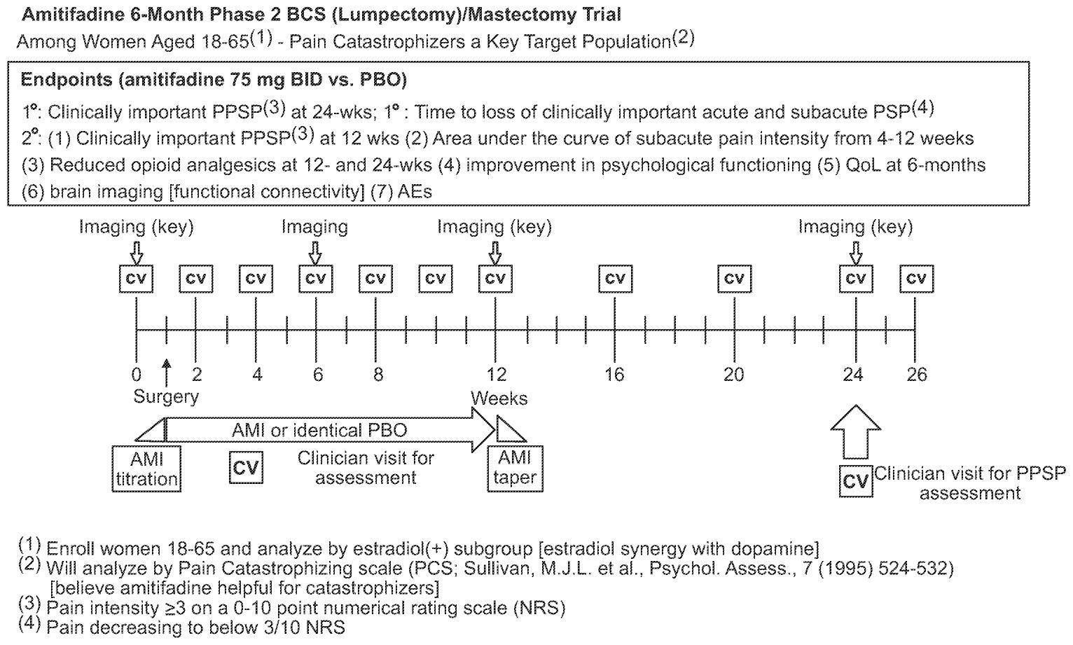

### Example 1: Prophylaxis or Reduction in Subacute and Chronic Pain Following Breast Surgery (FIG. 1)

### PROTOCOL SYNOPSIS Title of Study: A double-blind, placebo-controlled randomized trial of amitifadine in breast cancer surgery Estimated Number of Study Centers and Countries/Regions: 25, USA

Study Phase: II

Research Hypothesis: Treatment with amitifadine compared to placebo is associated with a reduced incidence of clinically important persistent post-surgical pain (PPSP) at 6 months following breast cancer surgery (BCS) or with a faster time to recovery from clinically important acute/subacute pain within the first 3 months following BCS.

Primary Objective: Generate data on the efficacy, safety, and tolerability of amitifadine in the prevention of chronic pain and the treatment of acute/subacute pain associated with BCS.

Study Design: Participants are women undergoing either mastectomy or lumpectomy for the treatment of breast cancer. They are enrolled prior to their surgery, and randomized to daily amitifadine treatment beginning one week before surgery and continuing for 3 months after surgery. Participants are followed for an additional 3 months.

Study Procedures: Once patients agree to participate in the study by signing the informed consent document, a medical and psychosocial history is taken and a physical examination is performed. Screened and eligible patients are asked to return one week before their surgery when they are randomized to double-blind treatment with placebo or amitifadine. The double-blind treatment lasts until 3 months following surgery, during which patients are seen every 2 weeks. A scheduling window of +/−3 days is allowed. The presence of any spontaneously reported side effect or adverse event is carefully documented. Reasons for premature discontinuation, including intolerable side effects, are recorded.

All concomitant medications taken during the study are recorded in the electronic case report form, along with dosage information and start and stop dates. Patients requiring excluded drugs are discontinued from the study. Medication management and clinical ratings are performed by the study clinicians.

Patients are randomly assigned using a 1:1 allocation ratio to amitifadine or matching placebo. The dosage regimen for patients in the amitifadine group is as follows: novel multimodal composition amitifadine 75 mg once daily in the morning beginning 7 days prior to surgery. Four days prior to surgery, give 75 mg once in the morning and once in the evening for a total daily dose of 150 mg/day through the day prior to surgery. Restart in recovery room or as soon as the patient can safely receive oral medication with amitifadine 75 mg bid for 12 weeks then discontinue. During the discontinuation week, administer 3 days of amitifadine 75 mg bid, then 4 days of once daily 75 mg. After 4 days, discontinue. Patients in the control group receive identical placebo instead of amitifadine 75 mg, and given according to the same regimen.

Patients may receive concomitant medications as follows:

Intraoperative— Standard anesthetic techniques are anticipated, including all appropriate medications for induction, maintenance of a patent airway, maintenance of anesthesia and pain control, and recovery.

Perioperative—Patient-controlled analgesia with morphine and acetaminophen are allowed for the duration of the hospital stay. Supplementary oral opioids are made available on demand for breakthrough pain.

Post-operative—For the first 3 months following surgery, rescue medication is limited to a maximum of 2,600 mg/day of acetaminophen (i.e., up to 650 mg qid), and the only permitted concomitant medication is stable use of a benzodiazepine or related drug for insomnia.

Following cessation of double-blind treatment, certain analgesic and psychiatric medications and dosages are permitted if necessary (a tiered protocol is provided to investigators).

Utilization of all pain medication is carefully noted throughout the pre-, peri-, and post-operative period. Subjects unable to tolerate the study medication are withdrawn from the trial. Every effort is made to encourage patients to comply with this dosage regimen and to take all study medications as instructed. All patients are instructed to return any excess medication at each visit. A pill count is done to corroborate the study drug record. Protocol violation is defined as less than 80% compliance by pill count.

Duration of Study: Including screening and baseline assessments, the study period is approximately 200 days, with an estimated enrollment period of 18 months.

Number of Subjects per Group: At baseline, 350 subjects are randomized: 175 to the amitifadine group and 175 to the matching placebo group.

Study Population: The study population includes women between the ages of 18 and 65 years of age scheduled for BCS. Exclusion criteria consists of the following:
American Society of Anesthesiologists status greater than II.
Emergency surgery (decision to operate made on day of surgery).
Preoperative renal failure (eGFR <60 mL/min).
Participants with impaired hepatic function (Child-Pugh score A and higher).
Chronic pain medication other than APAP and non-steroidal anti-inflammatory drugs.
Symptoms of neuropathy due to diabetes Type I or Type II.
Chronic neurologic conditions, including Parkinson's disease, Alzheimer's disease, and other conditions associated with dementia.
Significant other medical diseases such as congestive heart failure, coronary or peripheral vascular disease, chronic obstructive lung disease, or malignancy.
History of glaucoma or narrow angle glaucoma.
Presence of undiagnosed skin lesions or history of melanoma.
Presence of severe cardiovascular or pulmonary disease, bronchial asthma, renal, hepatic, or endocrine disease.
History of myocardial infarction with residual cardiac arrhythmia.
History of gastrointestinal bleeding or peptic ulcer.
Diagnosis of current depression (assessed via BDI, total >28 are excluded) or psychiatric disorder requiring treatment, or such a diagnosis in the previous 6 months.
Use of therapeutic doses of antidepressant medications (i.e., tricyclic antidepressants SSRIs, SNRIs, DNRIs).
Concurrent use of any drugs for neuropathic pain e.g., antiepileptics or SNRIs.
Current use of recreational drugs or recent history of alcohol abuse (pattern of drinking having social, financial, or physical consequences) or drug abuse (urine screening).
Current use of cannabinoids.
Current use of opioid pain medications.
Use of MAOls, currently or within the past 2 weeks.
Use of any of the following drugs: levodopa, bromocriptine, linezolid, metoclopramide, phenothiazines, promethazine/codeine, isoniazid, rifampin, pyrazinamide.
Oral iron supplementation.
Currently taking dopaminergic drugs.
In the judgment of the investigator, unable or unwilling to follow protocol and instructions.
For those receiving MRI: intra-axial implants (e.g., spinal cord stimulators or pumps), all exclusion criteria for MR safety: any metallic implants, pacemaker, brain or skull abnormalities, tattoos on large body parts, and claustrophobia.
Pregnancy or inability to use an effective method of birth control in sexually active men and women while taking the study drug and for one week thereafter. Barrier contraceptives (condoms or diaphragm) with spermicide, intrauterine devices (IUD's), hormonal contraceptives, oral contraceptive pills, surgical sterilization, and complete abstinence are examples of effective methods of contraception.
Following laboratory abnormalities: liver function tests (SGOT/SGPT) greater than twice the upper limit of normal; unexplained anemia (Hgb 13.5 to 17.5 g/dL for men, 12.0 to 15.5g/dL for women); evidence of renal insufficiency (creatinine >upper limit of normal) or any other abnormality that the principal investigator feels puts the participant at risk during the study.
History of chronic opioid use for pain management.
Any medical condition that in the investigator's judgment may prevent the individual from completing the study or put the individual at undue risk.
Limited understanding of numerical scoring scales.
Previous participation in other trials investigating analgesic agents in previous three months.

Investigational Product(s), Dose and Mode of Administration, Duration of Treatment with Investigational Product(s): Amitifadine 75 mg tablets and matching placebo for a maximum exposure of 100 days.

Study Assessments and Primary Endpoints: The primary outcome measures are daily and weekly ratings of average pain intensity using a 0-10 numerical rating scale (NRS).

Secondary Outcomes Include:
(1) Clinically important PPSP (0_3 on a 0-10 point NRS) at 12-wks
(2) Area under the curve of acute/subacute pain intensity from 4-12 wks.
(3) Reduced opioid analgesics at 12- and 24-wks
(4) At baseline and monthly for the entire trial: Brief Pain Inventory pain interference scale; Hospital Anxiety and Depression Scale; Short-Form McGill Pain Questionnaire-2; and Breast Cancer Pain Questionnaire (BCPQ); and SF-36
(5) Functional MRI brain imaging [ROI: Insula/Nucleus Accumbens/medial Prefrontal Cortex measuring functional connectivity/reward (DA)] [baseline, 6-wks, 12-wks, 24-wks]
(6) Adverse events

Statistical Methods: The intention-to-treat (ITT) sample includes all patients who are randomized. The safety sample includes those randomized patients who received at least one dose of double-blind study medication as indicated on the dosing record. The modified ITT sample includes those patients in the safety sample who have provided at least one pain rating post-randomization; this sample is used for the primary analyses.

The primary analyses involve testing the differences between the amitifadine and placebo groups in (1) the proportions of patients with clinically important PPSP at 6 months following surgery (i.e., 3 on 0-10 NRS of average pain intensity during the previous week); and (2) the time to loss of clinically important acute/subacute pain during the first 3 months following surgery (i.e., 3 on a 0-10 NRS for the weekly mean of average daily pain intensity). For each of these tests, a p-value of 0.025 two-tailed is considered a statistically significant difference.

FIG. 1 is a clinical study timeline titled: Amitifadine 6-Month Phase 2 BCS (Lumpectomy)/Mastectomy Trial—Among Women Aged 18-65 (Enroll women 18-65 and analyze by estradiol(+) subgroup (estradiol synergy with dopamine))— Pain Catastrophizers a Key Target Population (Will analyze by Pain Catastrophizing Scale (PCS: Sullivan, M. J. L. et al., Psycho!. Assess., 7 (1995) 524-532)— believe amitifadine helpful for catastrophizers). Endpoints (amitifadine 75 mg BID vs. PBO). Primary: Clinically important PPSP at 24 weeks; Primary: Time to loss of clinically important acute and subacute PSP. Pain intensity on a 0-10 point numerical rating scale (NRS). Pain decreasing to below 3/10 NRS. Secondary: (1) Clinically important PPSP at 12-wks; (2) Area under the curve of subacute pain intensity from 4-12 weeks; (3) Reduced opioid analgesics at 12- and 24-wks; (4) improvement in psychological functioning; (5) QoL at 6-months; (6) brain imaging (functional connectivity); (7) AEs. FIG. 1 further shows a timeline from 0 to 26 weeks, with clinician visit for assessment (CV) indicated, for example, at weeks 0, 2, 4, 6, 8, 10, 12, 16, 20, 24, and 26 weeks. Surgery is indicated at 1 week. CV for PPSP assessment is indicated at 24 weeks. Amitifidine (AMI) titration is indicated gradually from week 0 to week 1, then AMI or identical PBO from week 1 to week 12, followed by AMI taper from week 12 to week 13, for example. Imaging is indicated, for example, at week 0, week 6, week 12 (key), and week 24 (key).

### Example 2: Reduction in Chronic Pain Following Cardiac Surgery

A trial is designed to test the hypothesis that patients receiving novel multimodal composition, e.g., amitifadine for three months exhibit a significant reduction in clinically meaningful pain at six months after cardiac surgery compared to the control group. A placebo-controlled, double blind clinical trial is conducted with 350 patients (175 in test group, 175 in control group).

Inclusion criteria The patients provide informed consent to participate in the trial Men and women, aged 18-65 years receiving a first-time sternotomy for any cardiac surgery

Exclusion criteria
American Society of Anesthesiologists status greater than II.
Emergency surgery (decision to operate made on day of surgery).
Preoperative renal failure (eGFR <60 mL/min).
Participants with impaired hepatic function (Child-Pugh score A and higher).
Chronic pain medication other than APAP and non-steroidal anti-inflammatory drugs.
Symptoms of neuropathy due to diabetes Type I or Type II.
Chronic neurologic conditions, including Parkinson's disease, Alzheimer's disease, and other conditions associated with dementia.
Significant other medical diseases such as congestive heart failure, coronary or peripheral vascular disease, chronic obstructive lung disease, or malignancy.
History of glaucoma or narrow angle glaucoma.
Presence of undiagnosed skin lesions or history of melanoma.
Presence of severe cardiovascular or pulmonary disease, bronchial asthma, renal, hepatic, or endocrine disease.
History of myocardial infarction with residual cardiac arrhythmia.
History of gastrointestinal bleeding or peptic ulcer.
Diagnosis of current depression (assessed via BDI, total >28 are excluded) or psychiatric disorder requiring treatment, or such a diagnosis in the previous 6 months.
Use of therapeutic doses of antidepressant medications (i.e., tricyclic antidepressants SSRIs, SNRIs, DNRIs.
Concurrent use of any drugs for neuropathic pain e.g., antiepileptics or SNRIs.
Current use of recreational drugs or recent history of alcohol abuse (pattern of drinking having social, financial, or physical consequences) or drug abuse (urine screening).
Current use of cannabinoids.
Current use of opioid pain medications.
Use of MAOIs, currently or within the past 2 weeks.
Use of any of the following drugs: levodopa, bromocriptine, linezolid, metoclopramide, phenothiazines, promethazine/codeine, isoniazid, rifampin, pyrazinamide.
Oral iron supplementation.
Currently taking dopaminergic drugs.
In the judgment of the investigator, unable or unwilling to follow protocol and instructions.
For those receiving MRI: intra-axial implants (e.g., spinal cord stimulators or pumps), all exclusion criteria for MR safety: any metallic implants, pacemaker, brain or skull abnormalities, tattoos on large body parts, and claustrophobia.
Pregnancy or inability to use an effective method of birth control in sexually active men and women while taking the study drug and for one week thereafter. Barrier contraceptives (condoms or diaphragm) with spermicide, intrauterine devices (IUD's), hormonal contraceptives, oral contraceptive pills, surgical sterilization, and complete abstinence are examples of effective methods of contraception.
Following laboratory abnormalities: liver function tests (SGOT/SGPT) greater than twice the upper limit of normal; unexplained anemia (Hgb 13.5 to 17.5 g/dL for men, 12.0 to 15.5g/dL for women); evidence of renal insufficiency (creatinine >upper limit of normal) or any other abnormality that the principal investigator feels puts the participant at risk during the study.
History of chronic opioid use for pain management.
Any medical condition that in the investigator's judgment may prevent the individual from completing the study or put the individual at undue risk.
Limited understanding of numerical scoring scales.
Previous participation in other trials investigating analgesic agents in previous three months.

The primary outcome in the trial is the proportion of patients with clinically meaningful pain at six months after cardiac surgery. Meaningful pain is defined as a pain score on a numeric rating scale of greater than 3 out of a maximum score of 10, indicating moderate to severe pain intensity, after a functional assessment of three maximal coughs. Specifically, the Primary Outcome Measure for the trial is as follows: Numerical Rating Scale (NRS) pain score/Incidence of PPSP (around sternotomy incision site) following 3 maximal coughs (>3/10 in 0-10 numerical rating scale - where 0=no pain, 10=maximum pain; Time Frame: 6 months post-sternotomy).

Secondary Outcome Measures Include:

Total opioid consumption at 24 hours post-surgery [Time Frame: 24 hours post-surgery

Visual Analog Scale (VAS) scores at 24 hrs. post-surgery, at rest and following 3 maximal coughs [Time Frame: 24 hours post-surgery]

Sedation (including pCO2) and nausea scores at 24 hours post-surgery [Time Frame: 24 hours post-surgery]

Acute Adverse events [Time Frame: First 48 hours]

Chronic Adverse events [Time Frame: 6 months post-surgery]

Time to extubation [Time Frame: Post-op recovery period]

Length of stay in intensive care and hospital [Time Frame: Post-operative - acute]

28 day mortality [Time Frame: 28 days post-surgery]

Neuropathic pain score [Time Frame: 3 and 6 months post-surgery] using DN4 scale.

S-LANSS (Short form Leeds Assessment of Neuropathic Symptoms and Signs)

Quality of Life [Time Frame: 6 months] EQ-5D validated scoring scale

Quantitative Sensory Testing (QST) [Time Frame: Pre op and post op at 72 hrs. and 3 months] Pain Pressure Thresholds (PPT) using algometry, both pre and post Diffuse Noxious Inhibitory Control (DNIC) Tactile and Pain Detection Thresholds with mechanical static stimulus using von Frey hairs (VFH) Dynamic assessment of temporal summation and secondary hyperalgesia with VFH

Psychological functioning (anxiety/catastrophizing/mood) [Time Frame: pre-op, 3 months, and 6 months post-surgery]

Brain imaging [ROI: Insula/Nucleus Accumbens/medial Prefrontal Cortex measuring functional connectivity/reward (DA)] [baseline, 3 and 6 months]

The dosage regimen for patients in the test group receiving novel multimodal composition, e.g., amitifadine is as follows: novel multimodal composition, e.g., amitifadine 75 mg once daily in the morning beginning 7 days prior to surgery. Four days prior to surgery, give 75 mg once in the morning and once in the evening for a total daily dose of 150 mg/day through the day prior to surgery. Restart in recovery room or as soon as the patient can safely receive PO meds with novel multimodal composition, e.g., amitifadine 75 mg BID for 12 weeks then discontinue. During the discontinuation week, administer 3 days of 75 mg BID novel multimodal composition, e.g., amitifadine, then 4 days of once daily 75 mg. After 4 days, discontinue. Patients in the control group receive identical placebo instead of novel multimodal composition, e.g., amitifadine 75 mg, and given according to the same regimen.

The patients may receive concomitant medications, including:

Intraoperative—Standard anesthetic techniques are anticipated, including all appropriate medications for induction, maintenance of a patent airway, maintenance of anesthesia and pain control, and recovery.

Perioperative—Patient-controlled analgesia with morphine and APAP is allowed for the duration of the hospital stay. Supplementary oral opioids are available on demand for breakthrough pain.

Post-operative—Medications as needed (recognizing exclusions).

Utilization of all pain medication is carefully noted throughout the pre-, peri-, and post-operative period.Example 3

### Novel Methods for Mesocorticolimbic Activation

The PFC is involved with emotional function, and cognition including executive function such as planning, attention, decision-making, goal-directed behavior, and working memory. Because the experience of pain can be modified by emotions and expectations, the PFC is involved in pain processing and is a key site in the chronicification of pain as part of the mesocorticolimbic system. The PFC can be divided into the orbitofrontal cortex (OFC) (including the ventrolateral orbital cortex), the ventrolateral PFC, the dorsolateral PFC (DLPFC), the caudal PFC, and the medial prefrontal cortex (mPFC). The mPFC is composed of granular cortical areas and agranular regions (anterior cingulate cortex, ACC; infralimbic cortex (IL-PFC); and the prelimbic cortex (PL-PFC). The DLPFC, in particular, is involved in modulation of pain catastrophizing.

It is known that the PFC provides top-down control (cortical) control of sensory and emotional processes. Imaging studies in animals and humans shows the PFC is involved in pain, however, the bidirectional interaction between the cortical (PFC) and the limbic (nucleus accumbens) areas remain to be fully understood. Animal and human functional and imaging studies suggest that PFC and NAc and the functional connectivity in-between are altered in chronic pain.

This study is designed to test the hypothesis that modulation of 5-HT2c-mediated activation of PFC pyramidal neurons produces antiallodynic and affective ameliorative effects in a rat model (spared nerve injury model) of persistent neuropathic pain. Afferent pyramidal PFC neurons are known to interact with accumbral medium spiny neurons (MSNs). PFC activation thus, may be hypothesized to reduce allodynia and the affective symptoms of pain including anxiety, depression-like symptoms, and anhedonia through this long-distance interaction. This pain-relieving function of the PFC is believed mediated by pyramidal glutamatergic neurons with afferent projections to the core of the NAc (PL-PFC) and medial shell of the NAc (IL-PFC). The data generated from the study validates another mechanism of action by which amitifadine inhibits or prevents transition from acute to subacute pain and/or transition from acute to chronic pain (by activating a wholly separate circuit for pain regulation with anti-allodynic and emotional and cognitive improving sequelae).

This study is the first opportunity to evaluate the difference between a full 5-HT2c agonist and amitifadine, a 5-HT2c partial agonist.

Materials and Methods

Animals. All procedures in this study are approved consistent with institutional animal care and use committee (IACUC) regulations to ensure minimal animal use and discomfort. Male Sprague Dawley rats are purchased from standard sources and kept at kept at controlled humidity, room temperature, and 12 h light/dark cycle. Food and water are made available ad libitum. Animals arrive to the animal facility targeted at a weight of 250-300 g and are given 1 week adjust to the new environment before the onset of any experiments.

Drugs. Ro 60-0175 fumarate, a research 5-HT2c full agonist is used as a comparator and may be purchased from Tocris Cookson (Bristol, UK). Ro 60-0175 fumarate is dissolved in water (3 mg/kg/mL, dose refers to the salt) and is injected systemically (i.p.) 15 min before behavioral assays. (-O-1-(3,4-dichlorophenyl)-3-azabicyclo[3.1.0]hexane (amitifadine as the hydrochloride salt) is supplied from Ethismos Research, Inc. and dissolved in 0.9% saline. NBQX (2,3-dioxo-6-nitro-7-sulfamoyl-benzo[f]quinoxaline) is an antagonist of the AMPA receptor and, therefore, a glutamate antagonist is used to evaluate glutamatergic effects.

SNI surgery. Use standard SNI surgery technique (Decosterd, I, et al. Spared nerve injury: an animal model of persistent peripheral neuropathic pain. Pain. 2000 August; 87(2):149-158.).

Whole-cell recordings. Somatic whole-cell recordings are made from pyramidal cells in the PL-PFC and IL-PFC and medium spiny neurons in the NAc. Use NBQX to block glutamatergic activity as control.

Animal behavioral tests. Animals used for behavior receive either amitifadine, Ro-60-0175, NBQX, or saline (control group) in the IL-PFC and PL-PFC. Behavioral tests in the PFC sites are done 2 weeks after amitifadine injection, and tests with stimulation in the NAc core/shell are done 6-8 weeks after dosing.

Mechanical allodynia test. Measure mechanical allodynia using standard (von Frey filament) technique.

Cold allodynia test. Measure cold allodynia using standard (acetone) technique.

Thermal hyperalgesia. Use standard technique (e.g., Hargreaves test).

Conditional Place Preference (CPP). Evaluate conditional place preference for amitifadine vs. Ro 60-0175 vs. NBQX vs. vehicle.

Sucrose Preference Test. Perform SPT using standard technique.

Forced Swim Test. Use Porsolt technique.

Locomotor activities. Use standard rat LMA method.

Statistics. Report results of behavioral experiments as mean±SEM. Use two-way ANOVA and Student t-tests as appropriate. Use Bonferroni correction for multiple comparisons. For all tests, a p-value 0.05 are considered statistically significant.

### SPECIFIC EMBODIMENTS

Representative embodiments of the present disclosure are set forth in the following paragraphs.

Paragraph 1. A method of treating a patient experiencing pain or expecting to experience pain, comprising administering to the patient, on a substantially daily basis, a therapeutically effective amount of amitifadine, (+)-1-(3,4-dichlorophenyl)-3-azabicyclo[3.1.0]hexane (IUPAC: (1R,5S)-(+)-1-(3,4-dichlorophenyl)-3-azabicyclo[3.1.0]hexane), or a pharmaceutically acceptable salt thereof, wherein administration of amitifadine is administered daily for a time period comprising 0-3 weeks prior to onset of the pain and about 4 to about 52 weeks after the onset of the pain.

Paragraph 2. The method of paragraph 1, wherein the amitifadine is administered daily beginning about 1-3 weeks prior to the onset of the pain or a stimulus that causes the pain and continued for a time period comprising about 4 weeks after the onset of the pain.

Paragraph 3. The method of paragraph 1, wherein the amitifadine is administered daily beginning about 1-3 weeks prior to the onset of the pain or a stimulus that causes pain and continued for a time period comprising about 12 weeks after the onset of the pain.

Paragraph 4. The method of any one of paragraphs 1-3, wherein the amitifadine is administered daily beginning about 2 weeks prior to the onset of the pain or a stimulus that causes the pain.

Paragraph 5. The method any one of paragraphs 1-4, wherein the administration of amitifadine is initiated substantially concomitantly with the onset of the pain or a stimulus that causes the pain.

Paragraph 6. The method of any one of paragraphs 1-5, wherein the amitifadine is administered orally.

Paragraph 7. The method of any one of paragraphs 1-6, wherein the amitifadine is administered once or twice daily.

Paragraph 8. The method of any one of paragraphs 1-7, wherein the amitifadine is administered in a daily dosage of about 10 to about 300 mg.

Paragraph 9. The method of any one of paragraphs 1-8, wherein the patient is experiencing or expecting to experience pain as a result of surgery.

Paragraph 10. The method of paragraph 9, wherein the surgery is soft tissue surgery.

Paragraph 11. The method of paragraph 10, wherein the soft tissue surgery is breast surgery.

Paragraph 12. The method of paragraph 10, wherein the soft tissue surgery is cardiovascular surgery.

Paragraph 13. The method of paragraph 9, wherein the surgery is orthopedic surgery.

Paragraph 14. The method of paragraph 13, wherein the orthopedic surgery is joint replacement surgery.

Paragraph 15. The method of paragraph 14, wherein the joint replacement surgery is hip, knee or shoulder replacement surgery.

Paragraph 16. The method of any one of paragraphs 9-15, wherein the patient is experiencing pre-operative pain.

Paragraph 17. The method of any one of paragraphs 9-16, wherein the amitifadine is administered daily beginning about 2 weeks prior to surgery up to about a day prior to surgery and continued beginning about a day after surgery.

Paragraph 18. The method of any one of paragraphs 9-17, wherein the administration of amitifadine is effective to prevent a transition from the pain to persistent post-surgical pain.

Paragraph 19. The method of any one of paragraphs 1-8, wherein the patient is experiencing the pain as a result of non-surgical trauma.

Paragraph 20. The method of paragraph 19, wherein the non-surgical trauma is a vehicular accident, a sports injury, or a military injury.

Paragraph 21. The method of any one of paragraphs 1-20, wherein the patient has a history of, or has symptoms of pain catastrophization comprising a maladaptive response to pain characterized by an experience of heightened pain intensity, increased disability, or difficulty disengaging from pain or a belief that pain will intensify and cannot be ameliorated.

Paragraph 22. The method of any one of paragraphs 1-20, wherein the patient has a history of, or is experiencing depression or anxiety.

Paragraph 23. The method of any one of paragraphs 1-22 wherein the patient presents with symptoms of cognitive impairment.

Paragraph 24. The method of any one of paragraphs 1-23, wherein the patient is experiencing anhedonia.

Paragraph 25. The method of any one of paragraphs 1-24, wherein the patient is an adult female.

Paragraph 26. The method of paragraph 25, wherein the female patient is pre-menopausal.

Paragraph 27. The method of any one of paragraphs 1-26, wherein the amitifadine is administered in the form of a pharmaceutically acceptable salt.

Paragraph 28. The method of paragraph 27, wherein the amitifadine is administered in the form of a hydrochloride salt.

Paragraph 29. A method of treating a patient experiencing chronic pain, comprising administering to the patient, on a daily basis, a therapeutically effective amount of amitifadine, (+)-1-(3,4-dichlorophenyI)-3-azabicyclo[3.1.0]hexane (IUPAC: (1R,5S)-(+)-1-(3,4-dichlorophenyl)-3-azabicyclo[3.1.0]hexane), or a pharmaceutically acceptable salt thereof.

Paragraph 30. The method of paragraph 29, wherein the amitifadine is administered daily for about 1-24 weeks.

Paragraph 31. The method of paragraph 1, wherein the amitifadine is administered for about 1 to about 52 weeks.

Paragraph 32. The method of any one of paragraphs 29-31, wherein the amitifadine is administered orally.

Paragraph 33. The method of any one of paragraphs 29-32, wherein the amitifadine is administered once or twice daily.

Paragraph 34. The method of any one of paragraphs 29-33, wherein the amitifadine is administered in a daily dosage of about 10 to about 300 mg or about 10 mg to about 200 mg.

Paragraph 35. The method of any one of paragraphs 29-34, wherein the patient is experiencing musculoskeletal pain.

Paragraph 36. The method of paragraph 35, wherein the musculoskeletal pain is chronic lower back pain.

Paragraph 37. The method of any one of paragraphs 29-34, wherein the patient is experiencing neuropathic pain.

Paragraph 38. The method of paragraph 37, wherein the neuropathic pain is chronic postherpetic neuralgia.

Paragraph 39. The method of any one of paragraphs 29-34, wherein the patient is experiencing centralized pain.

Paragraph 40. The method of paragraph 39, wherein the centralized pain is chronic

fibromyalgia.

Paragraph 41. The method of any one of paragraphs 29-34, wherein the patient is experiencing visceral pain.

Paragraph 42. The method of paragraph 41, wherein the visceral pain is chronic pelvic pain.

Paragraph 43. A method for activating neurons of the prefrontal cortex (PFC) in a patient experiencing pain or expecting to experience pain, comprising administering to the patient, on a substantially daily basis, a therapeutically effective amount of amitifadine, (+)-1-(3,4-dichlorophenyl)-3-azabicyclo[3.1.0]hexane (IUPAC: (1R,5S)-(+)-1-(3,4-dichlorophenyl)-3-azabicyclo[3.1.0]hexane), or a pharmaceutically acceptable salt thereof, wherein administration of amitifadine is administered daily for a time period comprising 0-3 weeks prior to onset of the pain and about 4 to about 52 weeks after the onset of the pain.

Paragraph 44. The method of any one of paragraphs 29-43, wherein the patient has a history of, or has symptoms of pain catastrophization comprising a maladaptive response to pain characterized by an experience of heightened pain intensity, increased disability, or difficulty disengaging from pain or a belief that pain will intensify and cannot be ameliorated.

Paragraph 45. The method of any one of paragraphs 29-44, wherein the patient has a history of, or is experiencing depression or anxiety.

Paragraph 46. The method of any one of paragraphs 29-45, wherein the patient presents with symptoms of cognitive impairment.

Paragraph 47. The method of any one of paragraphs 29-46, wherein the patient is experiencing anhedonia.

Paragraph 48. The method of any one of paragraphs 29-47, wherein the patient is an adult female.

Paragraph 49. The method of any one of paragraphs 29-48, wherein the amitifadine is administered in the form of a salt.

Paragraph 50. The method of paragraph 49, wherein the amitifadine is administered in the form of a hydrochloride salt.

Paragraph 51. The method of any one of paragraphs 1-50, wherein the patient is receiving concomitant opioid therapy.

Paragraph 52. The method of paragraph 28, wherein the hydrochloride salt is polymorph A.

Paragraph 53. The method of paragraph 50, wherein the hydrochloride salt is polymorph A.

Paragraph 54. The method of paragraph 43, wherein the amitifadine is administered in the form of a salt.

Paragraph 55. The method of paragraph 50, wherein the amitifadine is administered in the form of a hydrochloride salt.

Paragraph 56. The method of paragraph 55, wherein the hydrochloride salt is polymorph A.

All patent publications and non-patent publications are indicative of the level of skill of those skilled in the art to which this disclosure pertains. All these publications are herein incorporated by reference to the same extent as if each individual publication were specifically and individually indicated as being incorporated by reference.

Although the disclosure has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A method of treating a patient experiencing pain or expecting to experience pain, comprising administering to the patient, on a substantially daily basis, a therapeutically effective amount of amitifadine, (+)-1-(3,4-dichlorophenyl)-3-azabicyclo[3.1.0]hexane (IUPAC: (1R,5S)-(+)-1-(3,4-dichlorophenyl)-3-azabicyclo[3.1.0]hexane), or a pharmaceutically acceptable salt thereof, wherein administration of amitifadine is administered daily for a time period comprising 0-3 weeks prior to onset of the pain and about 4 to about 52 weeks after the onset of the pain.

2. The method of claim 1, wherein the amitifadine is administered daily beginning about 1-3 weeks prior to the onset of the pain or a stimulus that causes the pain and continued for a time period comprising about 4 weeks after the onset of the pain.

3. The method of claim 1, wherein the amitifadine is administered daily beginning about 1-3 weeks prior to the onset of the pain or a stimulus that causes pain and continued for a time period comprising about 12 weeks after the onset of the pain.

4. The method of claim 1, wherein the amitifadine is administered daily beginning about 2 weeks prior to the onset of the pain or a stimulus that causes the pain.

5. The method claim 1, wherein the administration of amitifadine is initiated substantially concomitantly with the onset of the pain or a stimulus that causes the pain.

6. The method of claim 1, wherein the amitifadine is administered orally.

7. The method of claim 1, wherein the amitifadine is administered once or twice daily.

8. The method of claim 1, wherein the amitifadine is administered in a daily dosage of about 10 to about 300 mg.

9. The method of claim 1, wherein the patient is experiencing or expecting to experience pain as a result of surgery.

10. The method of claim 9, wherein the surgery is soft tissue surgery.

11. The method of claim 10, wherein the soft tissue surgery is breast surgery.

12. The method of claim 10, wherein the soft tissue surgery is cardiovascular surgery.

13. The method of claim 9, wherein the surgery is orthopedic surgery.

14. The method of claim 13, wherein the orthopedic surgery is joint replacement surgery.

15. The method of claim 14, wherein the joint replacement surgery is hip, knee or shoulder replacement surgery.

16. The method of claim 9, wherein the patient is experiencing pre-operative pain.

17. The method of claim 9, wherein the amitifadine is administered daily beginning about 2 weeks prior to surgery up to about a day prior to surgery and continued beginning about a day after surgery.

18. The method of claim 9, wherein the administration of amitifadine is effective to prevent a transition from the pain to persistent post-surgical pain.

19. The method of claim 1, wherein the patient is experiencing the pain as a result of non-surgical trauma.

20. The method of claim 19, wherein the non-surgical trauma is a vehicular accident, a sports injury, or a military injury.

21. The method of claim 1, wherein the patient has a history of, or has symptoms of pain catastrophization comprising a maladaptive response to pain characterized by an experience of heightened pain intensity, increased disability, or difficulty disengaging from pain or a belief that pain will intensify and cannot be ameliorated.

22. The method of claim 1, wherein the patient has a history of, or is experiencing depression or anxiety.

23. The method of claim 1, wherein the patient presents with symptoms of cognitive impairment.

24. The method of claim 1, wherein the patient is experiencing anhedonia.

25. The method of claim 1, wherein the patient is an adult female.

26. The method of claim 25, wherein the female patient is pre-menopausal.

27. The method of claim 1, wherein the amitifadine is administered in the form of a pharmaceutically acceptable salt.

28. The method of claim 27, wherein the amitifadine is administered in the form of a hydrochloride salt.

29. A method of treating a patient experiencing chronic pain, comprising administering to the patient, on a daily basis, a therapeutically effective amount of amitifadine, (+)-1-(3,4-dichlorophenyl)-3-azabicyclo[3.1.0]hexane (IUPAC: (111,55)-(+)-1-(3,4-dichlorophenyl)-3-azabicyclo[3.1.0]hexane), or a pharmaceutically acceptable salt thereof.

30. The method of claim 29, wherein the amitifadine is administered daily for about 1-24 weeks.

31. The method of claim 1, wherein the amitifadine is administered for about 1 to about 52 weeks.

32. The method of claim 29, wherein the amitifadine is administered orally.

33. The method of claim 29, wherein the amitifadine is administered once or twice daily.

34. The method of claim 29, wherein the amitifadine is administered in a daily dosage of about 10 to about 300 mg or about 10 mg to about 200 mg.

35. The method of claim 29, wherein the patient is experiencing musculoskeletal pain.

36. The method of claim 35, wherein the musculoskeletal pain is chronic lower back pain.

37. The method of claim 29, wherein the patient is experiencing neuropathic pain.

38. The method of claim 37, wherein the neuropathic pain is chronic postherpetic neuralgia.

39. The method of claim 29, wherein the patient is experiencing centralized pain.

40. The method of claim 39, wherein the centralized pain is chronic fibromyalgia.

41. The method of claim 29, wherein the patient is experiencing visceral pain.

42. The method of claim 41, wherein the visceral pain is chronic pelvic pain.

43. A method for activating neurons of the prefrontal cortex (PFC) in a patient experiencing pain or expecting to experience pain, comprising administering to the patient, on a substantially daily basis, a therapeutically effective amount of amitifadine, (+)-1-(3,4-dichlorophenyl)-3-azabicyclo[3.1.0]hexane (IUPAC: (1R,5S)-(+)-1-(3,4-dichlorophenyl)-3-azabicyclo[3.1.0]hexane), or a pharmaceutically acceptable salt thereof, wherein administration of amitifadine is administered daily for a time period comprising 0-3 weeks prior to onset of the pain and about 4 to about 52 weeks after the onset of the pain.

44. The method of claim 29, wherein the patient has a history of, or has symptoms of pain catastrophization comprising a maladaptive response to pain characterized by an experience of heightened pain intensity, increased disability, or difficulty disengaging from pain or a belief that pain will intensify and cannot be ameliorated.

45. The method of claim 29, wherein the patient has a history of, or is experiencing depression or anxiety.

46. The method of claim 29, wherein the patient presents with symptoms of cognitive impairment.

47. The method of claim 29, wherein the patient is experiencing anhedonia.

48. The method of claim 29, wherein the patient is an adult female.

49. The method of claim 29, wherein the amitifadine is administered in the form of a salt.

50. The method of claim 49, wherein the amitifadine is administered in the form of a hydrochloride salt.

51. The method of claim 1, wherein the patient is receiving concomitant opioid therapy.

52. The method of claim 28, wherein the hydrochloride salt is polymorph A.

53. The method of claim 50, wherein the hydrochloride salt is polymorph A.

54. The method of claim 43, wherein the amitifadine is administered in the form of a salt.

55. The method of claim 54, wherein the amitifadine is administered in the form of a hydrochloride salt.

56. The method of claim 55, wherein the hydrochloride salt is polymorph A.
